(19) United States
(12) Patent Application Publication
(10) Pub. No.: US 2009/0149496 A1
(43) Pub. Date: Jun. 11, 2009

(54) INHIBITORS OF THE TASK-1 AND TASK-3 ION CHANNEL

(75) Inventors: Joachim BRENDEL, Bad Vilbel (DE); Heinz GOEGELEIN, Frankfurt am Main (DE); Klaus WIRTH, Frankfurt am Main (DE); Walter KAMM, Frankfurt am Main (DE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 12/252,516

(22) Filed: Oct. 16, 2008

Related U.S. Application Data
(63) Continuation of application No. PCT/EP2007/003293, filed on Apr. 13, 2007.

(30) Foreign Application Priority Data
Apr. 27, 2006 (DE) 102006019589.2
Oct. 20, 2006 (DE) 102006049527.6

(51) Int. Cl. A61K 31/47; A61K 31/4412; A61K 31/18; A61K 31/381; A61P 25/28; A61P 35/00; A61P 11/00; A61P 25/16; A61K 31/341; A61K 31/166; A61K 31/44

(52) U.S. Cl. 514/311; 514/351; 514/357; 514/604; 514/616; 514/438; 514/471

(57) ABSTRACT

The invention relates to the use of Kv1.5 inhibitors for the therapy or prophylaxis of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer.

The invention relates to the use of compounds of the formulae Ia, Ib, Ic, Id, Ie, If, Ig, Ih and/or Ij

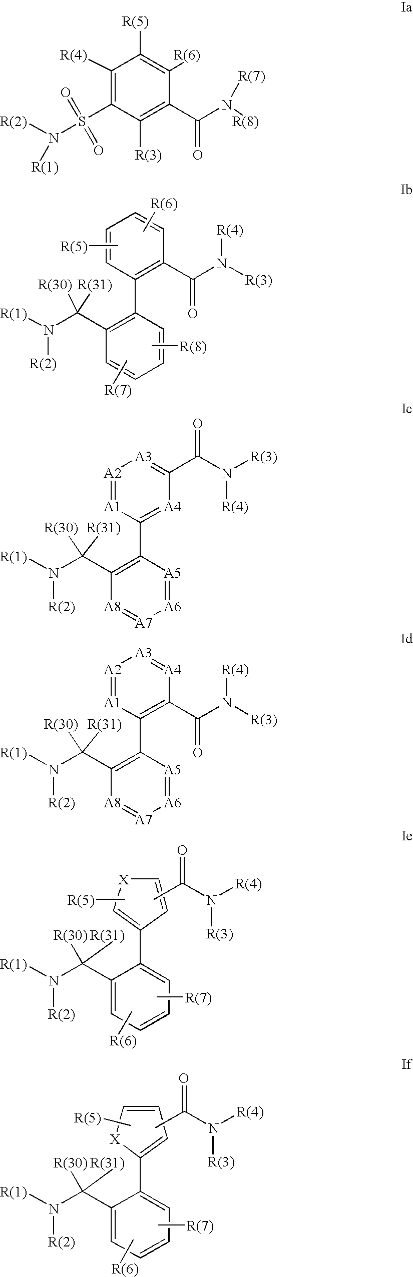

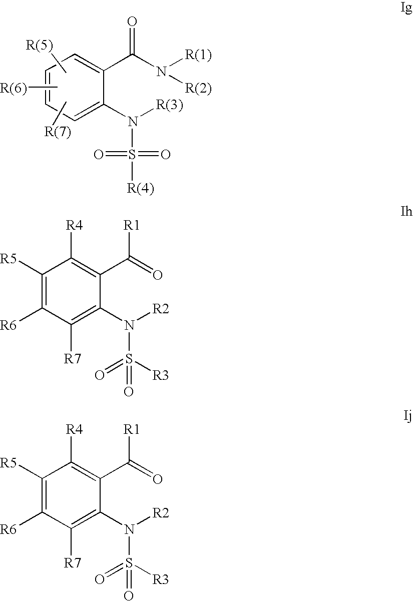

and/or physiologically compatible salts thereof for the production of a medicament for the therapy or prophylaxis of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer.

The compounds of the formulae Ia-Ij and/or physiologically compatible salts thereof inhibit so-called TASK potassium channels, especially the TASK-1 and/or TASK-3 subtypes.

Potassium channels are widespread membrane proteins which, owing to their influences on cell membrane potentials, play an important role in many physiological processes. Within the various classes of the potassium channels, a distinction is drawn on the basis of their molecular structure between three large groups which are characterized by the number of transmembrane domains (2, 4 or 6). The group of the potassium channels with four transmembrane segments is delimited from the two others in that their representatives each have two pore domains, which is why these channels are also referred to as K_{2P} channels [Coetzee W. J. et al; Molecular diversity of K+ channels; Ann. New York Acad. Sci. 1999 (868), 233-285]. In functional terms, K_{2P} channels are characterized in that the so-called “leak” or “background” streams flow through them, which play an important role for the resting membrane potential and hence the excitability of nerve or muscle cells.

A family which is of particular interest among the K_{2P} channels is that of the TASK channels, which were not discovered until the end of the 1990s and of which five representatives, TASK-1, TASK-2, TASK-3, TASK-4 and TASK-5, have now been described. Other terms used in the literature for the underlying genes are KCNK3 or K2P3.1 (=TASK-1), KCNK5 or K2P5.1 (=TASK-2), KCNK9 or K2P9.1 (=TASK-3), KCNK15 or K2P15.1 (=TASK-5) and KCNK17 or K2P17.1 (=TASK-4, TALK-2). The greatest homology within this family is possessed by the TASK-1 and TASK-3 channels with an amino acid identity of more than 50%. Dimerization of K_{2P} channels forms functional potassium channels with a total of four pore units. The streams which flow through these channels are referred to in the literature as IKso stream. In addition to a homodimerization of, for example, two TASK-1 or two TASK-3 proteins, heterodimerization of TASK-1 and TASK-3 is also possible in this context [Berg A. P., Talley E. M., Manger J. P., Bayliss D. A.; Motoneurons express Heteromeric TWIK-related acid-sensitive K+ (TASK) Channels containing TASK-1 (KCNK3) and TASK-3 (KCNK9) subunits; J. Neuroscience 2004 (24), 6693-6702].

The TASK channels are notable in particular for their very strong dependence upon the extracellular pH in the physiological range. The channels are inhibited at acidic pH and activated at alkaline pH. Owing to this pH dependence, the physiological function of a sensor which translates small changes in the extracellular pH to corresponding cellular signals is ascribed to the TASK channels [Duprat F., Lesage F., Fink M., Reyes R., Heurteaux C., Lazdunski M.; TASK, a human background K+ channel to sense external pH variations near physiological pH; EMBO J. 1997 (16), 5464-5471; Patel A. J., Honore E.; Properties and modulation of mammalian 2P domain K+ channels; Trends Neurosci. 2001 (24), 339-346].

TASK-1 is expressed in the brain and also in spinal ganglia and some peripheral tissues, for example pancreas, placenta, uterus, lung, heart, kidney, small intestine and stomach. In addition, TASK-1 has been detected in the chemosensitive cells of the brainstem and of the carotid bodies, and also the motor neurons of the hypoglossal nerve. TASK-3 is expressed mainly in the cerebellum [Medhurst A. D., Rennie G., Chapman C. G., Meadows H., Duckworth M. D., Kelsell R. E., Glober I. I., Pangalos M. N.; Distribution analysis of human two pore domain potassium channels in tissues of the central nervous system and periphery; Mol. Brain. Res. 2001 (86), 101-114].

Electrical currents which are caused by TASK-1 potassium channels have been detected in motor neurons of the hypoglossal nerve (a motor cranial nerve which possesses the most important function for the maintenance of the upper respiratory pathways) and locus coeruleus. It has been found that TASK-1 channels are involved in respiratory regulation in respiratory neurons of the brainstem, in carotid bodies and in motor neurons of the hypoglossal nerve, and also in neuroepithelial cells of the lung. In the event of inadequate respiration (hypoxia, hindered breathing) and in the event of physical stress, either via a rise in the CO₂ concentration and the resulting acidosis or via acidic metabolites (lactate), there is a lowering of the pH and hence a blockage of the pH-dependent TASK-1 channels. This depolarizes the cells, which leads to the activation of the neurons involved in the respiratory regulation [Buckler K. J., Williams B. A., Honore E.; An oxygen-, acid- and anaesthetic-sensitive TASK-like background potassium channel in rat arterial chemoreceptor cells; J. Physiol. 2000 (525), 135-142; Bayliss D. A., Talley E. M., Sirois J. E., Lei Q.; TASK-1 is a highly modulated pH-sensitive ‘leak’ K+ channel expressed in brainstem respiratory neurons; Respiration Physiology 2001 (129), 159-174].

An increase in the activity of chemosensitive neurons in conjunction with an activation of the motor neurons of the hypoglossal nerve through blockage of the TASK channel can stimulate respiration and simultaneously stabilize the upper respiratory pathways and protect them from collapse and occlusion. Moreover, snoring can be inhibited through the mechanism of stabilization of the upper respiratory pathways. The blockage of the TASK-1 ion channels can therefore find use for the treatment of respiratory disorders, for example of sleep apnea.

Obstructive sleep apneas arise through the reduced inspiratory pressure which is generated by the diaphragm and chest muscles in the course of inhalation into the upper respiratory pathways in the presence of contraction of the upper respiratory pathways. Constricted anatomic conditions of the upper respiratory pathways are present in the event of obesity (lipotrophy) and anatomic predisposition (e.g. retrognathia). In persons having this predisposition, the tone of the dilating muscle structure of the upper respiratory pathway muscle structure must always be increased in comparison to healthy persons in order to prevent collapse. The genioglossus muscle (a muscle at the base of the tongue) is the most important of the dilating muscles of the upper respiratory pathways; it is innervated by the hypoglossal nerve. While the muscle tone in the upper respiratory pathways is still sufficiently high in the wakeful state to prevent respiratory disorders, it falls greatly in sleep, such that it is too low in relation to the reduced inspiratory pressure. This disparity leads to the collapse of the upper respiratory pathways (obstructive apnea) during the inhalation. In the case of high constriction of the upper respiratory pathways and correspondingly high tissue pressure, a collapse can occur even during exhalation, i.e. without reduced pressure. An increase in the muscle tone of the upper respiratory pathways through the inventive Kv1.5 inhibitors therefore prevents obstructive apneas.

Snoring is generated by flow-related vibrations in the upper respiratory pathways. It arises in the case of excessively narrow upper respiratory pathways with simultaneously insufficient muscle tone of the upper respiratory pathways and hence has a close pathophysiological relationship to obstructive sleep apnea. Snoring can thus be regarded to some extent as a precursor of obstructive apnea. An increase in the muscle tone of the upper respiratory pathways through the inventive Kv1.5 inhibitors therefore prevents both snoring and obstructive sleep apneas.

Central apneas are caused by central disruptions of respiratory regulation. They are prevented by the simultaneously respiration-stimulating action of the inventive Kv1.5 inhibitors (effect on the minute volume).

TASK-1 channels are also present in smooth muscle cells of mesenterial and pulmonary arteries. In the latter, it is possible that they are involved in acidosis-induced pulmonary vasoconstriction [Gurney A. M., Osipenko O, N., MacMillan D., McFarlane K. M., Tate R. J., Kempsill F. E.; Two-pore domain K channel, TASK-1, in pulmonary artery smooth muscle cells; Circ. Res. 2003 (93), 957-964].

It has also been stated that TASK channels are involved in the secretion of adrenal gland hormones in the zona glomerulosa of the adrenal cortex [Czirjak G., Fischer T., Spät A., Lesage F., Enyedi P.; TASK (TWIK-related acid-sensitive K+ channel) is expressed in glomerulosa cells of rat adrenal cortex and inhibited by angiotensin II; Molecular Endocrinology 2000 (14), 863-874].

In cultivated granulosa cells of the cerebellum, it has been shown that genetic inactivation of TASK channels brings about neuroprotective action [Lauritzen I., Zanzouri M., Honoré E., Duprat F., Ehrengruber M. U., Lazdunski M., Patel A. J.; K+-dependent cerebellar granule neuron apoptosis—Role of Task leak K+ channels; J. Biol. Chem. 2003 (278), 32068-32076]. It has also been shown that TASK-1 channels are responsible for programmed cell death (apoptosis) in granulosa cells, and that the cell death can be prevented by blocking the TASK-3. It is therefore assumed that the development of specific inhibitors of the TASK-1/3 channels can mean a pharmacological strategy for the treatment of neurodegenerative disorders [Patel A. J., Lazdunski M., The 2P-domain K+ channels: role in apoptosis and tumorigenesis, Pflugers Arch. 2004 (448), 261-273].

The TASK-3 gene is amplified and overexpressed in various human carcinoma tissues, for example breast cancer, lung cancer, colon cancer and metastasizing prostate cancer [Mu D., Chen L., Zhang X., et al., Genomic amplification and oncogenic properties of the KCNK9 potassium channel gene, Cancer Cell 2003 (3), 297-302]. It has been found that the performance of a point mutation on TASK-3 switches off the channel function and simultaneously removes the tumor-forming function. It is therefore expected that TASK-3 antagonists might reduce the growth of various human cancers and thus constitute a new family of anticancer drugs [Pei L., Wiser O., Slavin A., Mu D., Powers S., Jan L. Y., Hoey T.; Oncogenic potential of TASK3 (Kcnk9) depends on K+ channel function; Proc. Natl. Acad. Sci. USA 2003 (100), 7803-7807].

In spite of the great physiological significance of the TASK channels, only very few pharmacological modulators of these channels are known to date in the literature. It has been stated that an activation of the TASK-1 channel can be achieved by therapeutic concentrations of the inhalative anesthetics halothane and isoflurane [Patel A. J., Honoré E., Lesage F., Fink M., Romey G., Lazdunski M.; Inhalational anesthetics activate two-pore-domain background K+ channels; Nature Neurosci. 1999 (2), 422-426]. The only known direct blockers of TASK-1 are the arachidonamides anandamide (an endogenous ligand of the cannabinoid receptor) and its methanandamide homolog, for which an IC50 value of 0.7 μm has been stated [Maingret F., Patel A. J., Lazdunski M., Honoré E.; The endocannabinoid anandamide is a direct and selective blocker of the background K+ channel TASK-1; EMBO J. 2001 (20), 47-54], and also doxapram, which is used for the treatment of respiratory disorders and for which an IC50 value of 0.4 μm has recently been described [Cotten J. F., Keshavaprasad B., Laster M. J., Eger E. I., Yost C. S.; The Ventilatory Stimulant Doxapram Inhibits TASK Tandem Pore (K_{2P}) Potassium Channel Function but Does Not Affect Minimum Alveolar Anesthetic Concentration; Anesth. Analg. 2006 (102) 779-785].

It has now been found that the compounds of the formulae Ia, Ib, Ic, Id, Ie, If, Ig, Ih and Ij are potent blockers of TASK channels, especially of the TASK-1 and TASK-3 subtypes. The compounds have to date only been known as blockers of Kv1.5 channels, which belong to the group of the potassium channels with 6 transmembrane domains and one pore domain (WO01/00573, WO01/025189, WO02/044137, WO02/046162, WO02/048131, WO02/087568, WO02/088073, WO02/100825). In view of the great structural differences between Kv1.5 channels and TASK channels, the action of these compounds known as Kv1.5 blockers on the TASK-1 and TASK-3 channel was surprising.

Owing to the TASK-1- and/or TASK-3-inhibitory properties, the compounds of the formulae Ia to Ij and/or their pharmaceutically compatible salts are suitable for the prevention and treatment of disorders which are caused by activation or by an activated TASK-1 and/or TASK-3, and also of disorders which have TASK-1- and/or TASK-3-related damage as a secondary cause.

The compounds of the formulae Ia, Ib, Ic, Id, Ie, If, Ig, Ih and Ij and/or physiologically compatible salts thereof can also be used for the treatment and prevention of disorders where the TASK-1 and/or TASK-3 is inhibited only partially, for example by using a lower dosage.

The compounds of the formulae Ia to Ij can be used in particular for the therapy or prophylaxis of respiratory disorders, sleep-related respiratory disorders, central and obstructive sleep apneas, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer.

In addition to the described blockage of TASK channels, the inhibition of other potassium channels, for example Kv channels, may also be relevant for the use of the inventive compounds of the formulae Ia to Ij for the therapy or prophylaxis of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia.

The present invention relates to the use of Kv1.5 inhibitors for producing a medicament for the therapy or prophylaxis of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer.

The invention relates to the use of compounds of the formulae Ia

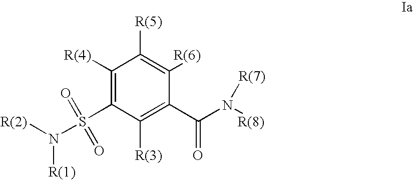

where R(8) is either a 1-indanyl radical of the formula II or a 2-indanyl radical of the formula III

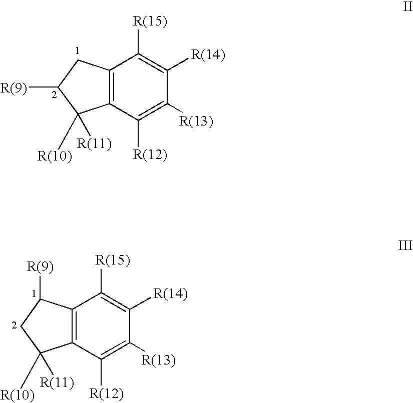

and in which:

### R(1) and R(2)

are each independently R(20)-CᵣH₂ᵣ
where one CH₂ group of the CᵣH₂ᵣ group may be replaced by —O—, —CH═CH—, —C═C—, —CO—, —CO—O—, —O—CO—, —S—, —SO—, —SO₂—, —NR(21)- or —CONR(21);
R(21) is hydrogen or alkyl having 1, 2 or 3 carbon atoms;
R(20) is H, CH₃, CH₂F, CHF₂, CF₃, C₂F₅, C₃F₇, cycloalkyl having 3, 4, 5, 6, 7 or 8 carbon atoms, NR(22)R(23), —CONR(22)R(23), —OR(24), —COOR(24), phenyl or an N-containing heterocycle having 1, 2, 3, 4, 5, 6, 7, 8 or 9 carbon atoms,
   where phenyl and the N-containing heterocycle are unsubstituted or substituted by one or two substituents selected from the group consisting of F, Cl, Br, I, CF₃, NO₂, CN, NH₂, OH, methyl, ethyl, hydroxymethyl, hydroxyethyl, methoxy, dimethylamino, sulfamoyl, methylsulfonyl and methylsulfonylamino;
   R(22) and R(23)
      are each independently hydrogen or alkyl having 1, 2, 3 or 4 carbon atoms;
   or
   R(22) and R(23)
      together are a chain of 4 or 5 methylene groups of which one CH₂ group may be replaced by —O—, —S—, —NH—, —N(methyl)— or —N(benzyl)-;
   R(24) is hydrogen alkyl having 1, 2 or 3 carbon atoms;
r is zero, 1, 2, 3, 4, 5, 6, 7 or 8;
   or

### R(1) and R(2)

together are a chain of 4 or 5 methylene groups of which one CH₂ group may be replaced by —O—, —S—, —NH—, —N(methyl)- or —N(benzyl)-;

### R(3), R(4), R(5) and R(6)

are each independently hydrogen, F, Cl, Br, I, alkyl having 1, 2, 3, 4 or 5 carbon atoms, cycloalkyl having 3, 4, 5, 6, 7 or 8 carbon atoms, CN, CF₃, NO₂, OR(25) or NR(26)R(27);
R(25) is hydrogen, alkyl having 1, 2, 3 or 4 carbon atoms, a fluorinated alkyl radical of the formula —CₓH₂ₓCF_{y}H_{3-y} or phenyl,
   which is unsubstituted or substituted by one or two substituents selected from the group consisting of F, Cl, Br, I, CF₃, NO₂, CN, NH₂, OH, methyl, ethyl, methoxy, dimethylamino, sulfamoyl, methylsulfonyl and methylsulfonylamino;
   x is 0, 1, 2 or 3;
   y is 1, 2 or 3;
R(26) and R(27)
   are each independently hydrogen or alkyl having 1, 2, 3 or 4 carbon atoms;
or
R(26) and R(27)
   together are a chain of 4 or 5 methylene groups of which one CH₂ group may be replaced by —O—, —S—, —NH—, —N(methyl)- or —N(benzyl)-;
      R(7) is hydrogen or alkyl having 1, 2, 3 or 4 carbon atoms;
      R(9) is hydrogen, OR(28) or OCOR(28);
R(28) is hydrogen or alkyl having 1, 2, 3 or 4 carbon atoms;

### R(10) and R(11)

are each independently hydrogen or alkyl having 1, 2, 3 or 4 carbon atoms;

### R(12), R(13), R(14) and R(15)

are each independently hydrogen, F, Cl, Br, I, alkyl having 1, 2, 3, 4 or 5 carbon atoms, cycloalkyl having 3, 4, 5, 6, 7 or 8 carbon atoms, —CN, —CF₃, —C₂F₅, —C₃F₇, —N₃, —NO₂, —Y—CₛH₂, —R(29), phenyl, thienyl, furyl or an N-containing heterocycle having 1, 2, 3, 4, 5, 6, 7, 8 or 9 carbon atoms,
   where phenyl, thienyl, furyl and the N-containing heterocycle are unsubstituted or substituted by one or two substituents selected from the group consisting of F, Cl, Br, I, CF₃, NO₂, CN, NH₂, OH, methyl, ethyl, methoxy, dimethylamino, sulfamoyl, methylsulfonyl and methylsulfonylamino;
Y is —O—, —CO—, —CO—O—, —O—CO—, —S—, —SO—, —SO₂—, —O—SO₂—, —SO₂NR(30)-, —CONR(30)- or —NR(30)CO—, where the bond to the base structure is in each case via the atom on the left;
   R(30) is hydrogen or alkyl having 1, 2 or 3 carbon atoms;
s is zero, 1, 2, 3, 4, 5 or 6;
R(29) is hydrogen, methyl, CF₃, C₂H₅, C₃F₇, cycloalkyl having 3, 4, 5, 6, 7 or 8 carbon atoms, —OR(31), —COOR(31), —NR(32)R(33), —CONR(32)R(33), phenyl or an N-containing heterocycle having 1, 2, 3, 4, 5, 6, 7, 8 or 9 carbon atoms;
   where phenyl and the N-containing heterocycle are unsubstituted or substituted by one or two substituents selected from the group consisting of F, Cl, Br, I, CF₃, NO₂, CN, NH₂, OH, methyl, ethyl, methoxy, dimethylamino, sulfamoyl, methylsulfonyl and methylsulfonylamino;
   R(31) is hydrogen or alkyl having 1, 2 or 3 carbon atoms;
   R(32) and R(33)
      are each independently hydrogen or alkyl having 1, 2, 3 or 4 carbon atoms;
   or
   R(32) and R(33)
      together are a chain of 4 or 5 methylene groups of which one CH₂ group may be replaced by —O—, —S—, —NH—, —N(CH₃)— or —N(benzyl)-;
         and/or compounds of the formula Ib

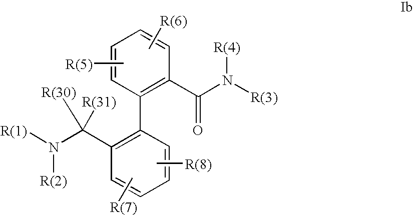

in which:

### R(1) is C(O)OR(9), SO₂R(10), COR(11), C(O)NR(12)R(13) or C(S)NR(12)R(13);

R(9) is CₓH₂ₓ—R(14);
   x is 0, 1, 2, 3 or 4,
   where x cannot be zero when R(14) is OR(15) or SO₂Me;
R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
   where phenyl, naphthyl, diphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(15) is alkyl having 1, 2, 3, 4 or 5 carbon atoms, cycloalkyl having 3, 4, or 6 carbon atoms, CF₃ or phenyl
      which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(10), R(11) and R(12)
   are each independently as defined for R(9);
R(13) is hydrogen, alkyl having 1, 2, 3 or 4 carbon atoms or CF₃;
   R(2) is hydrogen, alkyl having 1, 2, 3 or 4 carbon atoms or CF₃;

### R(3) is C_{y}H_{2y}—R(16);

y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
   where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
      where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         or

### R(3) is CHR(18)R(19);

R(18) is hydrogen or C_{z}H_{2z}—R(16) where R(16) is as defined above;
z is 0, 1, 2 or 3;
R(19) is COOH, CONH₂, CONR(20)R(21), COOR(22), CH₂OH;
   R(20) is hydrogen, alkyl having 1, 2, 3, 4 or 5 carbon atoms, CᵥH₂ᵥ—CF₃ or C_{w}H_{2w}-phenyl,
      where the phenyl ring is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   v is 0, 1, 2 or 3;
   w is 0, 1, 2 or 3;
   R(21) is hydrogen or alkyl having 1, 2, 3, 4 or 5 carbon atoms;
R(22) is alkyl having 1, 2, 3, 4 or 5 carbon atoms;
   R(4) is hydrogen, alkyl having 1, 2, 3, 4, 5 or 6 carbon atoms or CF₃;
   or

### R(3) and R(4)

together are a chain of 4 or 5 methylene groups of which one methylene group may be replaced by —O—, —S—, —NH—, —N(methyl)- or —N(benzyl)-;

### R(5), R(6), R(7) and R(8)

are each independently hydrogen, F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;

### R(30) and R(31)

are each independently hydrogen or alkyl having 1, 2 or 3 carbon atoms;
   or

### R(30) and R(31)

together are a chain of 2 methylene groups;
   and/or compounds of the formula Ic

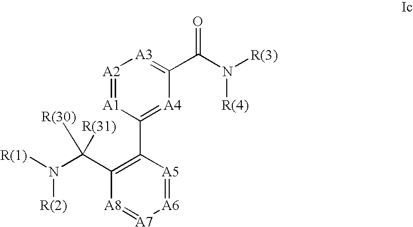

in which:

### A1, A2, A3, A4, A5, A6, A7 and A8

are each independently nitrogen, CH or CR5, where at least four of these groups are CH;

### R(1) is C(O)OR(9), SO₂R(10), COR(11), C(O)NR(12)R(13) or C(S)NR(12)R(13);

R(9), R(10), R(11) and R(12)
   are each independently CₓH₂ₓ—R(14);
   x is 0, 1, 2, 3 or 4;
   where x cannot be 0 when R(14) is OR(15) or SO₂Me;
   R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
      where phenyl, naphthyl, biphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(15) is alkyl having 1, 2, 3, 4 or 5 carbon atoms, cycloalkyl having 3, 4, 5 or 6 carbon atoms, CF₃ or phenyl
      which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
   R(2) is hydrogen, alkyl having 1, 2, 3 or 4 carbon atoms or CF₃;

### R(3) is C_{y}H_{2y}—R(16);

y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
   where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino,
   R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
      where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         or

### R(3) is CHR(18)R(19);

R(18) is hydrogen or C_{z}H_{2z}—R(16) where R(16) is as defined above;
z is 0, 1, 2 or 3;
R(19) is COOH, CONH₂, CONR(20)R(21), COOR(22) or CH₂OH;
   R(20) is hydrogen, alkyl having 1, 2, 3, 4 or 5 carbon atoms, CᵥH₂ᵥ—CF₃ or C_{w}H_{2w}-phenyl
   where the phenyl ring is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   v is 0, 1, 2 or 3;
   w is 0, 1, 2 or 3;
R(21) is hydrogen or alkyl having 1, 2, 3, 4 or 5 carbon atoms;
R(22) is alkyl having 1, 2, 3, 4 or 5 carbon atoms;
   R(4) is hydrogen, alkyl having 1, 2, 3, 4, 5 or 6 carbon atoms or CF₃;
   or

### R(3) and R(4)

together are a chain of 4 or 5 methylene groups of which one methylene group may be replaced by —O—, —S—, —NH—, —N(methyl)- or —N(benzyl)-;
   R(5) is F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino, where, in the case that a plurality of A1 to A8 radicals is defined as CR(5), the R(5) radicals are each defined independently;

### R(30) and R(31)

are each independently hydrogen or alkyl having 1, 2 or 3 carbon atoms;
   or

### R(30) and R(31)

together are a chain of 2 methylene groups;
   and/or compounds of the formula Id

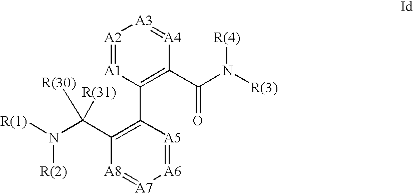

in which:
A1, A2, A3, A4, A5, A6, A7 and A8 are each independently nitrogen, CH or CR(5), where at least one of these groups is nitrogen and at least 4 of these groups are CH;

### R(1) is C(O)OR(9), SO₂R(10), COR(11), C(O)NR(12)R(13) or C(S)NR(12)R(13);

R(9), R(10), R(11) and R(12)
   are each independently CₓH₂ₓ—R(14);
   x is 0, 1, 2, 3 or 4;
   where x cannot be 0 when R(14) is OR(15) or SO₂Me;
   R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
      where phenyl, naphthyl, biphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(15) is alkyl having 1, 2, 3, 4 or 5 carbon atoms, cycloalkyl having 3, 4, 5 or 6 carbon atoms, CF₃ or phenyl which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
   R(2) is hydrogen, alkyl having 1, 2, 3 or 4 carbon atoms or CF₃;

### R(3) is C_{y}H_{2y}—R(16);

y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
   where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino,
   R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
      where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         or

### R(3) is CHR(18)R(19);

R(18) is hydrogen or C_{z}H_{2z}—R(16) where R(16) is as defined above;
z is 0, 1, 2 or 3;
R(19) is COOH, CONH₂, CONR(20)R(21), COOR(22) or CH₂OH;
   R(20) is hydrogen, alkyl having 1, 2, 3, 4 or 5 carbon atoms, CᵥH₂ᵥ—CF₃ or C_{w}H_{2w}-phenyl
      where the phenyl ring is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   v is 0, 1, 2 or 3;
   w is 0, 1, 2 or 3;
   R(21) is hydrogen or alkyl having 1, 2, 3, 4 or 5 carbon atoms;
   R(22) is alkyl having 1, 2, 3, 4 or 5 carbon atoms;
      R(4) is hydrogen, alkyl having 1, 2, 3, 4, 5 or 6 carbon atoms or CF₃;
      or

### R(3) and R(4)

together are a chain of 4 or 5 methylene groups of which one methylene group may be replaced by —O—, —S—, —NH—, —N(methyl)- or —N(benzyl)-;
   R(5) are each independently F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino, where, in the case that a plurality of A1 to A8 radicals is defined as CR(5), the R(5) radicals are each defined independently;

### R(30) and R(31)

are each independently hydrogen or alkyl having 1, 2 or 3 carbon atoms;
   or

### R(30) and R(31)

together are oxygen or a chain of 2 methylene groups;
   and/or compounds of the formula Ie or If

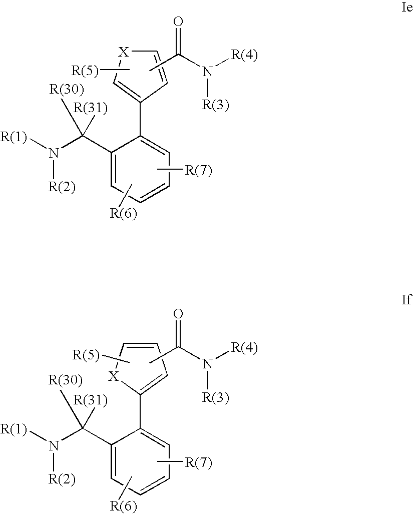

in which:
X is oxygen or sulfur;

### R(1) is C(O)OR(9), SO₂R(10), COR(11), C(O)NR(12)R(13) or C(S)NR(12)R(13);

R(9), R(10), R(11) and R(12)
are each independently CₓH₂ₓ—R(14);
x is 0, 1, 2, 3 or 4;
where x cannot be 0 when R(14) is OR(15) or SO₂Me;
R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
   where phenyl, naphthyl, biphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is alkyl having 1, 2, 3, 4 or 5 carbon atoms, cycloalkyl having 3, 4, 5 or 6 carbon atoms, CF₃ or phenyl which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
   R(2) is hydrogen, alkyl having 1, 2, 3 or 4 carbon atoms or CF₃;

### R(3) is C_{y}H_{2y}—R(16);

y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
   where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino,
   R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
      where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         or

### R(3) is CHR(18)R(19);

R(18) is hydrogen or C_{z}H_{2z}—R(16) where R(16) is as defined above;
z is 0, 1, 2 or 3;
R(19) is COOH, CONH₂, CONR(20)R(21), COOR(22) or CH₂OH;
   R(20) is hydrogen, alkyl having 1, 2, 3, 4 or 5 carbon atoms, CᵥH₂ᵥ—CF₃ or C_{w}H_{2w}-phenyl
      where the phenyl ring is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   v is 0, 1, 2 or 3;
   w is 0, 1, 2 or 3;
   R(21) is hydrogen or alkyl having 1, 2, 3, 4 or 5 carbon atoms;
   R(22) is alkyl having 1, 2, 3, 4 or 5 carbon atoms;
      R(4) is hydrogen, alkyl having 1, 2, 3, 4, 5 or 6 carbon atoms or CF₃;
      or

### R(3) and R(4)

together are a chain of 4 or 5 methylene groups of which one methylene group may be replaced by —O—, —S—, —NH—, —N(methyl)- or —N(benzyl)-;

### R(5), R(6) and R(7)

are each independently hydrogen, F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;

### R(30) and R(31)

are each independently hydrogen or alkyl having 1, 2 or 3 carbon atoms;
   or

### R(30) and R(31)

together are oxygen or a chain of 2 methylene groups;
   and/or compounds of the formula Ig

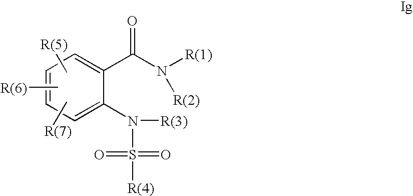

in which
R(1) is (CH₂)ₓ—R(8)
x is 0, 1, 2, 3, 4 or 5,
R(8) is phenyl, thienyl or furanyl,
   where phenyl, thienyl and furanyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
      R(2) is hydrogen or alkyl having 1, 2 or 3 carbon atoms;
      R(3) is hydrogen or alkyl having 1, 2 or 3 carbon atoms;
      R(4) is alkyl having 3, 4, 5, 6 or 7 carbon atoms, cycloalkyl having 3, 4, 5, 6 or 7 carbon atoms, phenyl, naphthyl or heteroaryl,
where phenyl and heteroaryl are unsubstituted or substituted by 1, 2 or 3 substituents selected from the group consisting of F, Cl, Br, I, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;

### R(5), R(6) and R(7)

are each independently F, Cl, Br, I, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
and/or compounds of the formula Ih

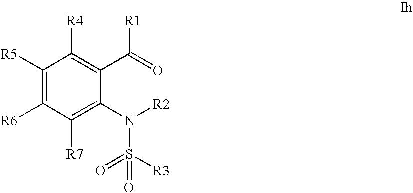

in which:

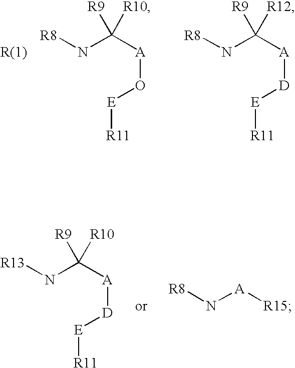

A is —CₙH₂ₙ—;
   n is 0, 1, 2, 3, 4 or 5;
O is oxygen;
D is a bond or oxygen;
E is —CₘH₂ₘ—;
   m is 0, 1, 2, 3, 4 or 5;
R(8) is hydrogen, alkyl having 1, 2, 3 or 4 carbon atoms or CₚH₂ₚ—R(14);
   p is 0, 1, 2, 3, 4 or 5;
   R(14) is cycloalkyl having 3, 4, 5 or 6 carbon atoms, aryl or heteroaryl,
      where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(9) is hydrogen or alkyl having 1, 2, 3, 4, 5 or 6 carbon atoms;
R(10) is hydrogen, alkyl having 1, 2, 3 or 4 carbon atoms, cycloalkyl having 3, 4, 5 or 6 carbon atoms, aryl or heteroaryl,
   where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(11) is cycloalkyl having 3, 4, 5 or 6 carbon atoms, phenyl, naphthyl, thienyl, furyl, pyridyl, pyrazinyl, pyridazinyl or pyrimidyl,
   where phenyl, naphthyl, thienyl, furyl, pyridyl, pyrazinyl, pyridazinyl and pyrimidyl are each unsubstituted or substituted by 1, 2 or 3 substituents selected from the group consisting of F, Cl, Br, I, CF₃, OCF₃, NO₂, CN, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(12) is alkyl having 1, 2, 3 or 4 carbon atoms, cycloalkyl having 3, 4, 5 or 6 carbon atoms, aryl or heteroaryl,
   where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is CₚH₂ₚ—R(14′);
   p is 0, 1, 2, 3, 4 or 5;
   R(14′) is cycloalkyl having 3, 4, 5 or 6 carbon atoms, tetrahydrofuranyl, tetrahydropyranyl, aryl or heteroaryl,
      where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is cycloalkyl having 3, 4, 5, 6, 7 or 8 carbon atoms;
   R(2) is hydrogen or alkyl having 1, 2, 3 or 4 carbon atoms;
   R(3) is alkyl having 3, 4, 5, 6 or 7 carbon atoms, cycloalkyl having 3, 4, 5 or 6 carbon atoms, phenyl or naphthyl,
where phenyl or naphthyl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

### R(4), R(5), R(6) and R(7)

are each independently hydrogen, F, Cl, Br, I, CF₃, OCF₃, OCHF₂, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   and/or compounds of the formula Ij

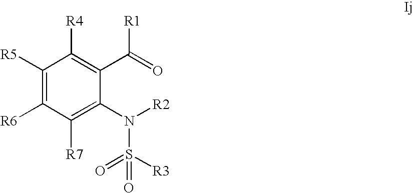

in which:

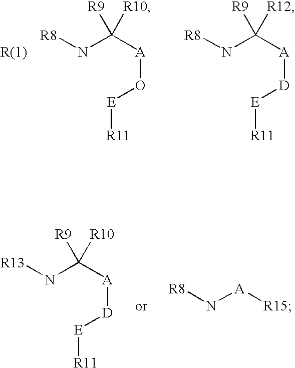

A is —CₙH₂ₙ—;
   n=0, 1, 2, 3, 4 or 5;
D is a bond or —O—;
   E is —Cₘ2₂ₘ—;
   m=0, 1, 2, 3, 4 or 5;
R(8) is hydrogen, alkyl having 1, 2, 3 or 4 carbon atoms or CₚH₂ₚ—R(14);
   p is 0, 1, 2, 3, 4 or 5;
   R(14) is phenyl, naphthyl or heteroaryl,
      where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(9) is hydrogen or alkyl having 1, 2, 3, 4, 5 or 6 carbon atoms;
R(10) is hydrogen, alkyl having 1, 2, 3 or 4 carbon atoms, phenyl, naphthyl or heteroaryl,
   where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(11) is cycloalkyl having 3, 4, 5 or 6 carbon atoms, phenyl, naphthyl, thienyl, furyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl,
   where phenyl, naphthyl, thienyl, furyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl are each unsubstituted or substituted by 1, 2 or 3 substituents selected from the group consisting of F, Cl, Br, I, CF₃, OCF₃, NO₂, CN, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(12) is alkyl having 1, 2, 3 or 4 carbon atoms, alkynyl having 1, 2, 3 or 4 carbon atoms, cycloalkyl having 3, 4, 5 or 6 carbon atoms, phenyl, naphthyl or heteroaryl,
   where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is CₚH₂ₚ—R(14);
   p is 0, 1, 2, 3, 4 or 5;
R(15) is cycloalkyl having 3, 4, 5, 6, 7 or 8 carbon atoms;
   R(2) is hydrogen or alkyl having 1, 2, 3 or 4 carbon atoms;
   R(3) is heteroaryl,
where heteroaryl is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

### R(4), R(5), R(6) and R(7)

are each independently hydrogen, F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   and/or physiologically compatible salts of the compounds of the formula Ia, Ib, Ic, Id, Ie, If, Ig, Ih or Ij for producing a medicament for the therapy or prophylaxis of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer.

Preference is given to the use of compounds of the formulae Ia to Ij and/or of a physiologically compatible salt thereof for the production of a medicament for the therapy or prophylaxis of respiratory disorders, especially sleep apneas, and cancer disorders.

A use of compounds of the formulae Ia to Ij and/or of a physiologically compatible salt thereof consists in the production of a medicament for the therapy or prophylaxis of respiratory disorders, in particular sleep-related respiratory disorders such as central and obstructive sleep apneas, upper airway resistance syndrome, Cheyne-Stokes respiration, snoring, disrupted central respiratory drive (sudden child death, postoperative hypoxia and apnea), muscle-related respiratory disorders, respiratory disorders after long-term ventilation, respiratory disorders during adaptation in high mountains, acute and chronic lung disorders with hypoxia and hypercapnia.

A further use of compounds of the formulae Ia to Ij and/or of a physiologically compatible salt thereof consists in the production of a medicament for the therapy or prophylaxis of respiratory disorders, in particular sleep-related respiratory disorders such as central and obstructive sleep apneas, Cheyne-Stokes respiration, snoring, disrupted central respiratory drive (sudden child death, postoperative hypoxia and apnea), muscle-related respiratory disorders, respiratory disorders after long-term ventilation, respiratory disorders during adaptation in high mountains, acute and chronic lung disorders with hypoxia and hypercapnia.

A preferred use of compounds of the formulae Ia to Ij and/or of a physiologically compatible salt thereof consists in the production of a medicament for the therapy or prophylaxis of sleep-related respiratory disorders such as central and obstructive sleep apneas, upper airway resistance syndrome and snoring.

Another preferred use of compounds of the formulae Ia to Ij and/or of a physiologically compatible salt thereof consists in the production of a medicament for the therapy or prophylaxis of sleep apneas, for example central and obstructive sleep apnea, and snoring.

A further use of compounds of the formulae Ia to Ij and/or of a physiologically compatible salt thereof consists in the production of a medicament for the therapy or prophylaxis of neurodegenerative disorders such as dementia, Alzheimer's disease, Parkinson's disease, Huntington's disease.

A further use of compounds of the formulae Ia to Ij and/or of a physiologically compatible salt thereof consists in the production of a medicament for the therapy or prophylaxis of cancer disorders, for example breast cancer, lung cancer, colon cancer and prostate cancer.

In one embodiment, the compounds of the formula Ia to Ij are used to produce a medicament for intravenous administration, especially to produce a medicament for intravenous administration for the therapy or prophylaxis of respiratory disorders, preferably sleep-related respiratory disorders such as central and obstructive sleep apneas, upper airway resistance syndrome and snoring, for example sleep-related respiratory disorders such as central and obstructive sleep apneas and snoring.

In a further embodiment, the compounds of the formula Ia to Ij are used to produce a medicament for oral administration, especially to produce a medicament for oral administration for the therapy or prophylaxis of respiratory disorders, preferably sleep-related respiratory disorders such as central and obstructive sleep apneas, upper airway resistance syndrome and snoring, for example sleep-related respiratory disorders such as central and obstructive sleep apneas and snoring.

In a further embodiment, the compounds of the formula Ia to Ij are used to produce a medicament for nasal administration, especially to produce a medicament for nasal administration for the therapy or prophylaxis of respiratory disorders, preferably sleep-related respiratory disorders such as obstructive sleep apneas, upper airway resistance syndrome and snoring, for example sleep-related respiratory disorders such as obstructive sleep apneas and snoring.

In one embodiment, compounds of the formula Ia are used, in which:

R(1) is hydrogen;

### R(2) is R(20)-CᵣH₂ᵣ;

R(20) is CH₃, CH₂F, CHF₂, CF₃, cycloalkyl having 3, 4, 5, 6, 7 or 8 carbon atoms, —CONR(22)R(23), —OR(24), —COOR(24) or phenyl
   which is unsubstituted or substituted by 1 or 2 substituents selected from the group consisting of F, Cl, Br, CF₃, NO₂, CN, OH, methyl, ethyl, hydroxymethyl, hydroxyethyl, methoxy, dimethylamino, sulfamoyl, methylsulfonyl and methylsulfonylamino;
   R(22) and R(23)
      are each independently hydrogen or alkyl having 1, 2, 3 or 4 carbon atoms;
   or
   R(22) and (R(23)
      together are a chain of 4 or 5 methylene groups of which one CH₂ group may be replaced by —O—, —S—, —NH—, —N(methyl)- or —N(benzyl)-;
   R(24) is hydrogen or alkyl having 1, 2 or 3 carbon atoms;
r is zero, 1, 2, 3, 4 or 5;

### R(3), R(4), R(5) and R(6)

are each independently hydrogen, F, Cl, Br, I, alkyl having 1, 2, 3, 4 or 5 carbon atoms, cycloalkyl having 3, 4, 5, 6, 7 or 8 carbon atoms, CN, CF₃, NO₂ or OR(25);
   R(25) is hydrogen, alkyl having 1, 2, 3 or 4 carbon atoms, a fluorinated alkyl radical of the formula —CₓH₂ₓCF_{y}H_{3-y} or phenyl which is unsubstituted or substituted by 1 or 2 substituents selected from the group consisting of F, Cl, Br, CF₃, NO₂, CN, OH, methyl, ethyl, methoxy, dimethylamino, sulfamoyl, methylsulfonyl and methylsulfonylamino;
      x is 0, 1, 2 or 3;
      y is 1, 2 or 3;
         R(7) is hydrogen or alkyl having 1, 2, 3 or 4 carbon atoms;
         R(8) is a 1-indanyl radical of the formula II;
         R(9) is hydrogen or OR(28);
R(28) is hydrogen or alkyl having 1, 2, 3 or 4 carbon atoms;

### R(10) and R(11)

are each independently hydrogen or alkyl having 1, 2, 3 or 4 carbon atoms;

### R(12), R(13), R(14) and R(15)

are each independently hydrogen, F, Cl, Br, I, alkyl having 1, 2, 3, 4 or 5 carbon atoms, cycloalkyl having 3, 4, 5, 6, 7 or 8 carbon atoms, CN, CF₃, —NO₂ or —Y—CₛH₂ₛ—R(29);
Y is —O—, —CO—, —CO—O—, —O—CO—, —S—, —SO—, —SO₂—, —O—SO₂—, —SO₂NR(30)-, —CONR(30)- or —NR(30)CO—, where the bond to the base structure is in each case via the atom on the left;
   R(30) is hydrogen or alkyl having 1, 2 or 3 carbon atoms;
s is zero, 1, 2, 3, 4 or 5;
R(29) is hydrogen, methyl, CF₃, —OR(31), —COOR(31), —NR(32)R(33), —CONR(32)R(33) or phenyl
   which is unsubstituted or substituted by 1 or 2 substituents selected from the group consisting of F, Cl, Br, CF₃, NO₂, CN, OH, methyl, ethyl, methoxy, dimethylamino, sulfamoyl, methylsulfonyl and methylsulfonylamino;
   R(31) is hydrogen or alkyl having 1, 2 or 3 carbon atoms;
   R(32) and R(33)
      are each independently hydrogen or alkyl having 1, 2, 3 or 4 carbon atoms;
   or
   R(32) and R(33)
      together are a chain of 4 or 5 methylene groups of which one CH₂ group may be replaced by —O—, —S—, —NH—, —N(CH₃)— or —N(benzyl)-;
         and/or compounds of the formula Ib in which:

### R(1) is C(O)OR(9), SO₂R(10), COR(11) or C(O)NR(12)R(13);

R(9) is CₓH₂ₓ—R(14);
   x is 0, 1, 2, 3 or 4,
   where x cannot be 0 when R(14) is OR(15);
   R(14) is alkyl having 1, 2, 3 or 4 carbon atoms, cycloalkyl having 3, 4, 5, 6, 7, 8 or 9 carbon atoms, CF₃, C₂F₅, OR(15), phenyl, furyl, thienyl or an N-containing heteroaromatic having 1, 2, 3, 4, 5, 6, 7, 8 or 9 carbon atoms,
      where phenyl, furyl, thienyl and the N-containing heteroaromatic are each unsubstituted or substituted by 1, 2 or 3 substituents selected from the group consisting of F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(15) is alkyl having 1, 2, 3, 4 or 5 carbon atoms, cycloalkyl having 3, 4, 5 or 6 carbon atoms, CF₃ or phenyl
      which is unsubstituted or substituted by 1, 2 or 3 substituents selected from the group consisting of F, Cl, Br, CF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(10), R(11) and R(12)
   are each independently as defined for R(9);
R(13) is hydrogen, alkyl having 1, 2, 3 or 4 carbon atoms or CF₃;
   R(2) is hydrogen, alkyl having 1, 2, 3 or 4 carbon atoms or CF₃;

### R(3) is C_{y}H_{2y}—R(16);

y is 0, 1, 2, 3 or 4,
where y cannot be 0 when R(16) is OR(17);
R(16) is alkyl having 1, 2, 3 or 4 carbon atoms, cycloalkyl having 3, 4, 5, 6, 7, 8 or 9 carbon atoms, CF₃, C₂F₅, OR(17), phenyl, furyl, thienyl or an N-containing heteroaromatic having 1, 2, 3, 4, 5, 6, 7, 8 or 9 carbon atoms,
   where phenyl, furyl, thienyl and the N-containing heteroaromatic are each unsubstituted or substituted by 1, 2 or 3 substituents selected from the group consisting of F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(17) is alkyl having 1, 2, 3, 4 or 5 carbon atoms, cycloalkyl having 3, 4, 5 or 6 carbon atoms, CF₃, phenyl or 2-, 3- or 4-pyridyl,
   where phenyl or 2-, 3- or 4-pyridyl are unsubstituted or substituted by 1, 2 or 3 substituents selected from the group consisting of F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
      or

### R(3) is CHR(18)R(19);

R(18) is hydrogen or C_{z}H_{2z}—R(16) where R(16) is as defined above
z is 0, 1, 2 or 3;
R(19) is CONH₂, CONR(20)R(21), COOR(22), CH₂OH;
   R(20) is hydrogen, alkyl having 1, 2, 3, 4 or 5 carbon atoms, CᵥH₂ᵥ—CF₃ or C_{w}H_{2w}-phenyl,
      where the phenyl ring is unsubstituted or substituted by 1, 2 or 3 substituents selected from the group consisting of F, Cl, Br, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   v is 0, 1, 2 or 3;
   w is 0, 1, 2 or 3;
   R(21) is hydrogen or alkyl having 1, 2, 3, 4 or 5 carbon atoms;
   R(22) is alkyl having 1, 2, 3, 4 or 5 carbon atoms;
      R(4) is hydrogen, alkyl having 1, 2, 3, 4, 5 or 6 carbon atoms or CF₃;

### R(5), R(6), R(7) and R(8)

are each independently hydrogen, F, Cl, Br, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;

### R(30) and R(31)

are each independently hydrogen or alkyl having 1, 2 or 3 carbon atoms;
   or

### R(30) and R(31)

together are a chain of two methylene groups;
   and/or compounds of the formula Ic in which:

### A1, A2, A3, A4, A5, A6, A7 and A8

are each independently nitrogen, CH or CR(5), where at most one of these groups is nitrogen and at least 5 of these groups are CH;

### R(1) is C(O)OR(9), SO₂R(10), COR(11) or C(O)NR(12)R(13);

R(9), R(10), R(11) and R(12)
   are each independently CₓH₂ₓ—R(14);
   x is 0, 1, 2, 3 or 4;
   R(14)
      is alkyl having 1, 2, 3 or 4 carbon atoms, cycloalkyl having 3, 4, 5, 6, 7, 8 or 9 carbon atoms, CF₃, phenyl, naphthyl, biphenylyl, furyl, thienyl or an N-containing heteroaromatic having 1, 2, 3, 4, 5, 6, 7, 8 or 9 carbon atoms,
      where phenyl, naphthyl, biphenylyl, furyl, thienyl and the N-containing heteroaromatic are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen;
   R(2) is hydrogen or methyl;

### R(3) is C_{y}H_{2y}—R(16);

y is 0, 1, 2, 3 or 4;
   where y cannot be 0 when R(16) is OR(17);
R(16) is alkyl having 1, 2, 3, 4, 5 or 6 carbon atoms, cycloalkyl having 3, 4, 5, 6, 7, 8 or 9 carbon atoms, CF₃, OR(17), SO₂Me, phenyl, naphthyl, furyl, thienyl or an N-containing heteroaromatic having 1, 2, 3, 4, 5, 6, 7, 8 or 9 carbon atoms,
   where phenyl, naphthyl, furyl, thienyl and the N-containing heteroaromatic are each unsubstituted or substituted by 1, 2 or 3 substituents selected from the group consisting of F, Cl, Br, I, CF₃, NO₂, OCF₃, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
      where phenyl or 2-, 3- or 4-pyridyl are each unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         R(4) is hydrogen, alkyl having 1 or 2 carbon atoms;
         R(5) is F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;

### R(30) and R(31)

are each independently hydrogen or methyl;
   and/or compounds of the formula (Id) in which:
   A1, A2, A3, A4, A5, A6, A7 and A8 are each independently nitrogen, CH or CR(5), where at least one of these groups is and at most two of these groups are nitrogen and at least four of these groups are CH;

### R(1) is C(O)OR(9), SO₂R(10), COR(11) or C(O)NR(12)R(13)

R(9), R(10), R(11) and R(12)
   are each independently CₓH₂ₓ—R(14);
   x is 0, 1, 2, 3 or 4;
   where x cannot be 0 when R(14) is OR(15);
   R(14) is alkyl having 1, 2, 3 or 4 carbon atoms, cycloalkyl having 3, 4, 5, 6, 7, 8 or 9 carbon atoms, CF₃, OR(15), phenyl, naphthyl, biphenylyl, furyl, thienyl or an N-containing heteroaromatic having 1, 2, 3, 4, 5, 6, 7, 8 or 9 carbon atoms,
      where phenyl, naphthyl, biphenylyl, furyl, thienyl and the N-containing heteroaromatic are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(15) is alkyl having 1, 2, 3, 4 or 5 carbon atoms, cycloalkyl having 3, 4, 5 or 6 carbon atoms, CF₃ or phenyl which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
   R(2) is hydrogen, alkyl having 1, 2, 3 or 4 carbon atoms or CF₃;

### R(3) is C_{y}H_{2y}—R(16);

y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8 or 9 carbon atoms, CF₃, OR(17), SO₂Me, phenyl, naphthyl, furyl, thienyl or an N-containing heteroaromatic having 1, 2, 3, 4, 5, 6, 7, 8 or 9 carbon atoms,
   where phenyl, naphthyl, furyl, thienyl and the N-containing heteroaromatic are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
      where phenyl or 2-, 3- or 4-pyridyl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         or

### R(3) is CHR(18)R(19);

R(18) is hydrogen or C_{z}H_{2z}—R(16) where R(16) is as defined above;
   z is 0, 1, 2 or 3;
R(19) is CONH₂, CONR(20)R(21), COOR(22) or CH₂OH;
   R(20) is hydrogen, alkyl having 1, 2, 3, 4 or 5 carbon atoms, CᵥH₂ᵥ—CF₃ or C_{w}H_{2w}-phenyl,
      where the phenyl ring is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   v is 0, 1, 2 or 3;
   w is 0, 1, 2 or 3;
   R(21) is hydrogen or alkyl having 1, 2, 3, 4 or 5 carbon atoms;
   R(22) is alkyl having 1, 2, 3, 4 or 5 carbon atoms;
      R(4) is hydrogen, alkyl having 1, 2, 3, 4, 5 or 6 carbon atoms or CF₃;
      R(5) are each independently F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;

### R(30) and R(31)

are each independently hydrogen or alkyl having 1, 2 or 3 carbon atoms;
   or

### R(30) and R(31)

are or a chain of 2 methylene groups;
   and/or compounds of the formula Ie or If, in which:
   X is oxygen or sulfur;

### R(1) is C(O)OR(9) or COR(11);

R(9) and R(11)
   are each independently CₓH₂ₓ—R(14);
   x is 0, 1, 2 or 3;
   R(14) is cycloalkyl having 5 or 6 carbon atoms or phenyl
      where phenyl is unsubstituted or substituted by 1 or 2 substituents selected from the group consisting of F, Cl, Br, I, CF₃, OCF₃, OH, alkyl having 1, 2 or 3 carbon atoms or alkoxy having 1 or 2 carbon atoms;
         R(2) is hydrogen;

### R(3) is C_{y}H_{2y}—R(16);

y is 0, 1 or 2;
R(16) is alkyl having 1, 2 or 3 carbon atoms, cycloalkyl having 5 or 6 carbon atoms, phenyl or pyridyl,
   where phenyl and pyridyl are each unsubstituted or substituted by 1, 2 or 3 substituents selected from the group consisting of F, Cl, CF₃, alkyl having 1, 2 or 3 carbon atoms and alkoxy having 1 or 2 carbon atoms;
      R(4) is hydrogen;

### R(5), R(6) and R(7)

are each independently hydrogen, F, Cl, alkyl having 1, 2 or 3 carbon atoms or alkoxy having 1 or 2 carbon atoms;

### R(30) and R(31)

are each hydrogen;
   and/or compounds of the formula Ig in which:
   R(1) is (CH₂)ₓ—R(8)
x is 1 or 2;
R(8) is phenyl, thienyl or furanyl,
   where phenyl, thienyl and furanyl are each unsubstituted or substituted by 1, 2 or 3 substituents selected from the group consisting of F, Cl, Br, CF₃, OCF₃, CN, COOMe, CONH₂, COMe, alkyl having 1, 2 or 3 carbon atoms, alkoxy having 1, 2 or 3 carbon atoms, sulfamoyl, methylsulfonyl and methylsulfonylamino;
      R(2) is hydrogen or alkyl having 1 or 2 carbon atoms;
      R(3) is hydrogen;
      R(4) is phenyl
where phenyl is unsubstituted or substituted by 1, 2 or 3 substituents selected from the group consisting of F, Cl, Br, CF₃, OCF₃, COMe, alkyl having 1, 2 or 3 carbon atoms and alkoxy having 1, 2 or 3 carbon atoms;

### R(5), R(6) and R(7)

are each independently F, Cl, Br, CF₃, OCF₃, CN, COOMe, CONH₂, COMe, alkyl having 1, 2 or 3 carbon atoms, alkoxy having 1, 2, 3 or 4 carbon atoms, dimethylamino, sulfamoyl, methylsulfonyl and methylsulfonylamino;
   and/or compounds of the formula Ih in which:

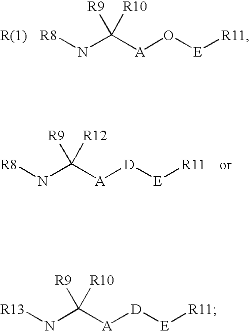

A is —CₙH₂ₙ—;
   n is 0, 1, 2 or 3;
O is oxygen;
D is a bond or oxygen;
E is —CₘH₂ₘ—;
   m is 0, 1, 2 or 3;
R(8) is hydrogen or alkyl having 1, 2, 3 or 4 carbon atoms;
R(9) is hydrogen or alkyl having 1, 2, 3, 4, 5 or 6 carbon atoms;
R(10) is hydrogen, alkyl having 1, 2, 3 or 4 carbon atoms or cycloalkyl having 3, 4 or 5 carbon atoms,
R(11) is cycloalkyl having 3, 4, 5 or 6 carbon atoms, phenyl or pyridyl,
   where phenyl and pyridyl are each unsubstituted or substituted by 1, 2 or 3 substituents selected from the group consisting of F, Cl, Br, CF₃, OCF₃, NO₂, CN, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(12) is alkyl having 1, 2, 3 or 4 carbon atoms, cycloalkyl having 3, 4 or 5 carbon atoms, aryl or heteroaryl,
   where aryl and heteroaryl are each unsubstituted or substituted by 1, 2 or 3 substituents selected from the group consisting of F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is CₚH₂ₚ—R(14′);
   p is 0, 1, 2, 3, 4 or 5;
   R(14′) is cycloalkyl having 3, 4, 5 or 6 carbon atoms, tetrahydrofuranyl, tetrahydropyranyl, aryl or heteroaryl,
      where aryl and heteroaryl are each unsubstituted or substituted by 1, 2 or 3 substituents selected from the group consisting of F, Cl, Br, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
         R(2) is hydrogen or alkyl having 1 or 2 carbon atoms;
         R(3) is alkyl having 3, 4 or 5 carbon atoms or phenyl;
where phenyl is unsubstituted or substituted by 1, 2 or 3 substituents selected from the group consisting of F, Cl, Br, CF₃, OCF₃, NO₂, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

### R(4), R(5), R(6) and R(7)

are each independently hydrogen, F, Cl, Br, CF₃, OCF₃, OCHF₂, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   and/or compounds of the formula Ij in which:

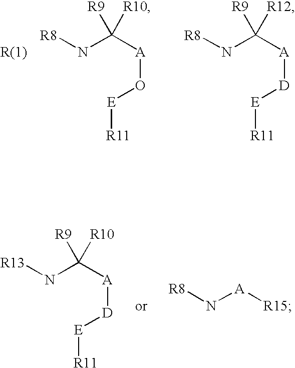

A is —CₙH₂ₙ—;
   n=0, 1, 2, 3, 4 or 5;
D is a bond or —O—;
E is —CₘH₂ₘ—;
   m=0, 1, 2, 3, 4 or 5;
R(8) is hydrogen, alkyl having 1, 2, 3 or 4 carbon atoms or CₚH₂ₚ—R(14);
   p is 0, 1, 2, 3, 4 or 5;
   R(14) is phenyl, naphthyl or heteroaryl,
      where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(9) is hydrogen or alkyl having 1, 2, 3, 4, 5 or 6 carbon atoms;
R(10) is hydrogen, alkyl having 1, 2, 3 or 4 carbon atoms, phenyl, naphthyl or heteroaryl,
where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(11) is cycloalkyl having 3, 4, 5 or 6 carbon atoms, phenyl, naphthyl, thienyl, furyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl,
   where phenyl, naphthyl, thienyl, furyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl are each unsubstituted or substituted by 1, 2 or 3 substituents selected from the group consisting of F, Cl, Br, I, CF₃, OCF₃, NO₂, CN, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(12) is alkyl having 1, 2, 3 or 4 carbon atoms, alkynyl having 1, 2, 3 or 4 carbon atoms, cycloalkyl having 3, 4, 5 or 6 carbon atoms, phenyl, naphthyl or heteroaryl,
   where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is CₚH₂ₚ—R(14);
   p is 0, 1, 2, 3, 4 or 5;
R(15) is cycloalkyl having 3, 4, 5, 6, 7 or 8 carbon atoms;
   R(2) is hydrogen or alkyl having 1, 2, 3 or 4 carbon atoms;
   R(3) is heteroaryl
where heteroaryl is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;

### R(4), R(5), R(6) and R(7)

are each independently hydrogen, F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
   and/or physiologically compatible salts of the compounds of the formula Ia, Ib, Ic, Id, Ie, If, Ig, Ih or Ij.

In a further embodiment, compounds of the formula Ia are used.

In a further embodiment, compounds of the formula Ib are used.

In a further embodiment, compounds of the formula Ic are used.

In a further embodiment, compounds of the formula Id are used.

In a further embodiment, compounds of the formula Ie are used.

In a further embodiment, compounds of the formula If are used.

In a further embodiment, compounds of the formula Ig are used.

In a further embodiment, compounds of the formula Ih are used.

In a further embodiment, compounds of the formula Ij are used.

Particular preference is given to the use of the compounds of the formula Ia, Ib, Ic, Id, Ie, If, Ih and Ij.

In one embodiment, preference is given to compounds of the formula Ia in which R(8) is a 1-indanyl radical of the formula II, for example a 1-indanyl of the formula II in which R9, R10, R11, R12, R13, R14 and R15 are each hydrogen.

In a further embodiment, preference is given to compounds of the formula Ia in which R(1) is hydrogen.

In a further embodiment, preference is given to compounds of the formula Ia in which R(2) is R(20)-CᵣH₂ᵣ where R(20) is CH₃, CH₂F, CHF₂, CF₃, cycloalkyl having 3, 4, 5, 6, 7 or 8 carbon atoms, —CONR(22)R(23), —OR(24), —COOR(24) or phenyl which is unsubstituted or substituted by 1 or 2 substituents selected from the group consisting of F, Cl, Br, CF₃, NO₂, CN, OH, methyl, ethyl, hydroxymethyl, hydroxyethyl, methoxy, dimethylamino, sulfamoyl, methylsulfonyl and methylsulfonylamino and r is zero, 1, 2, 3, 4 or 5; particular preference is given to compounds of the formula Ia in which R(2) is R(20)-CᵣH₂ᵣ— where R(20) is CH₃ and r is 4.

In a further embodiment, preference is given to compounds of the formula Ia in which R(3), R(4), R(5) and R(6) are each independently hydrogen, F, Cl, Br, I, alkyl having 1, 2, 3, 4 or 5 carbon atoms, cycloalkyl having 3, 4, 5, 6, 7 or 8 carbon atoms, CN, CF₃, NO₂ or OR(25); particular preference is given to compounds of the formula Ia in which R(3), R(4), R(5) and R(6) are each independently hydrogen or alkyl having 1, 2 or 3 carbon atoms, for example methyl; especially preferred are compounds of the formula Ia in which R(3), R(4) and R(5) are each hydrogen and R(6) is methyl.

In a further embodiment, preference is given to compounds of the formula Ia in which R(7) is hydrogen or alkyl having 1, 2, 3 or 4 carbon atoms, for example hydrogen.

In one embodiment, preference is given to compounds of the formula Ib in which R(1) is C(O)OR(9) or COR(11), where R(9) and R(11) are each CₓH₂ₓ—R(14) where x is 0, 1, 2 or 3 and R(14) is cycloalkyl having 5 or 6 carbon atoms or phenyl, where phenyl is unsubstituted or substituted by 1 or 2 substituents selected from the group consisting of F, Cl, CF₃, OCF₃, alkyl having 1, 2 or 3 carbon atoms and alkoxy having 1 or 2 carbon atoms; particular preference is given to compounds of the formula Ib in which R(1) is C(O)OR(9) or COR(11), where R(9) and R(11) are each CₓH₂ₓ—R(14) where x is 1 or 2 and R(14) is phenyl, where phenyl is unsubstituted or substituted by one substituent selected from the group consisting of alkoxy having 1 or 2 carbon atoms, for example methoxy.

In a further embodiment, preference is given to compounds of the formula Ib in which R(2) is hydrogen.

In a further embodiment, preference is given to compounds of the formula Ib in which R(3) is C_{y}H_{2y}—R(16) where y is 0, 1, 2 or 3 and R(16) is alkyl having 1, 2 or 3 carbon atoms, cycloalkyl having 5 or 6 carbon atoms, CF₃, OR17, phenyl or pyridyl, where R17 is hydrogen and where phenyl and pyridyl are each unsubstituted or substituted by 1 or 2 substituents selected from the group consisting of F, Cl, CF₃, OCF₃, alkyl having 1, 2 or 3 carbon atoms and alkoxy having 1 or 2 carbon atoms; particular preference is given to compounds of the formula Ib in which R(3) is C_{y}H_{2y}—R(16) where y is 1, 2 or 3 and R(16) is OR17, phenyl or pyridyl, where R17 is hydrogen and where phenyl and pyridyl are each unsubstituted or substituted by 2 substituents selected from the group consisting of F and Cl, for example F.

In a further embodiment, preference is given to compounds of the formula Ib in which R(4) is hydrogen or alkyl having 1 or 2 carbon atoms, for example hydrogen or methyl.

In a further embodiment, preference is given to compounds of the formula Ib in which R(5), R(6), R(7) and R(8) are each independently hydrogen, F, Cl, CF₃, alkyl having 1, 2 or 3 carbon atoms or alkoxy having 1 or 2 carbon atoms; particular preference is given to compounds of the formula Ib in which R(5) is hydrogen or Cl and R(6), R(7) and R(8) are each hydrogen.

In a further embodiment, preference is given to compounds of the formula Ib in which R(30) and R(31) are each hydrogen.

In one embodiment, preference is given to compounds of the formula Ic in which A1, A2, A3, A4, A5, A6, A7 and A8 are each independently nitrogen, CH or CR(5), where at most one of these groups is nitrogen and at least five of these groups are CH; particular preference is given to compounds of the formula Ic in which A1 is CR(5) where R(5) is alkyl having 1, 2 or 3 carbon atoms, for example methyl, and A2, A3, A4, A5, A6, A7 and A8 are each CH.

In a further embodiment, preference is given to compounds of the formula Ic in which R(1) is C(O)OR(9) or COR(11), where R(9) and R(11) are each independently CₓH₂ₓ—R(14) where x is 0, 1, 2 or 3 and R(14) is cycloalkyl having 5 or 6 carbon atoms or phenyl, where phenyl is unsubstituted or substituted by 1 or 2 substituents selected from the group consisting of F, Cl, Br, I, CF₃, OCF₃, OH, alkyl having 1, 2 or 3 carbon atoms or alkoxy having 1 or 2 carbon atoms; particular preference is given to compounds of the formula Ic in which R(1) is COR(11), where R(11) is CₓH₂ₓ—R(14) where x is 2 and R(14) is phenyl, where phenyl is substituted by one substituent selected from the group consisting of alkoxy having 1 or 2 carbon atoms, for example methoxy.

In a further embodiment, preference is given to compounds of the formula Ic in which R(2) is hydrogen.

In a further embodiment, preference is given to compounds of the formula Ic in which R(3) is C_{y}H_{2y}—R(16) where y is 0, 1, 2, 3 or 4 and R(16) is alkyl having 1, 2 or 3 carbon atoms, cycloalkyl having 5 or 6 carbon atoms, phenyl or pyridyl, where phenyl and pyridyl are each unsubstituted or substituted by 1, 2 substituents selected from the group consisting of F, Cl, CF₃, OCF₃, alkyl having 1, 2 or 3 carbon atoms or alkoxy having 1 or 2 carbon atoms; particular preference is given to compounds of the formula Ic in which R(3) is C_{y}H_{2y}—R(16) where y is 4 and R(16) is alkyl having 1, 2 or 3 carbon atoms, for example methyl.

In a further embodiment, preference is given to compounds of the formula Ic in which R(4) is hydrogen.

In a further embodiment, preference is given to compounds of the formula Ic in which R(30) and R(31) are each hydrogen.

In one embodiment, preference is given to compounds of the formula Id in which one of A1, A4 and A7 is nitrogen and the other of A1, A4 and A7 in each case and A2, A3, A5, A6 and A8 are each independently CH or CR(5), where at least five of these groups are CH; particular preference is given to compounds of the formula Id in which one of A1, A4 and A7 is nitrogen and the other of A1, A4 and A7 in each case and A2, A3, A5, A6 and A8 are each CH.

In a further embodiment, preference is given to compounds of the formula Id in which R(1) is C(O)OR(9) or COR(11), where R(9) and R(11) are each independently CₓH₂ₓ—R(14) where x is 0, 1, 2 or 3 and R(14) is cycloalkyl having 5 or 6 carbon atoms or phenyl, where phenyl is unsubstituted or substituted by 1 or 2 substituents selected from the group consisting of F, Cl, Br, I, CF₃, OCF₃, OH, alkyl having 1, 2 or 3 carbon atoms or alkoxy having 1 or 2 carbon atoms; particular preference is given to compounds of the formula Id in which R(1) is C(O)OR(9) or COR(11), where R(9) and R(11) are each independently CₓH₂ₓ—R(14) where x is 1, 2 or 3 and R(14) is phenyl.

In a further embodiment, preference is given to compounds of the formula Id in which R(2) is hydrogen.

In a further embodiment, preference is given to compounds of the formula Id in which R(3) is C_{y}H_{2y}—R(16) where y is 0, 1, 2, 3 or 4 and R(16) is alkyl having 1, 2 or 3 carbon atoms, cycloalkyl having 3, 4, 5 or 6 carbon atoms, phenyl or pyridyl, where phenyl and pyridyl are each unsubstituted or substituted by 1 or 2 substituents selected from the group consisting of F, Cl, CF₃, alkyl having 1, 2 or 3 carbon atoms and alkoxy having 1 or 2 carbon atoms; particular preference is given to compounds of the formula Id in which R(3) is C_{y}H_{2y}—R(16) where y is 1, 2 or 4 and R(16) is alkyl having 1, 2 or 3 carbon atoms, for example methyl, cycloalkyl having 3 carbon atoms, phenyl or pyridyl, where phenyl and pyridyl are each unsubstituted or substituted by two substituents selected from the group consisting of F or Cl, for example F.

In a further embodiment, preference is given to compounds of the formula Id in which R(4) is hydrogen.

In a further embodiment, preference is given to compounds of the formula Id in which R(30) and R(31) are each hydrogen.

In a further embodiment, preference is given to compounds of the formula Ie in which X is oxygen or sulfur, for example sulfur.

In a further embodiment, preference is given to compounds of the formula Ie in which R(1) is C(O)OR(9) or COR(11), where R(9) and R(11) are each independently CₓH₂ₓ—R(14) where x is 0, 1, 2, 3 or 4 and R(14) is alkyl having 1, 2 or 3 carbon atoms, cycloalkyl having 5 or 6 carbon atoms or phenyl, where phenyl is unsubstituted or substituted by 1 or 2 substituents selected from the group consisting of F, Cl, Br, I, CF₃, OCF₃, OH, alkyl having 1, 2 or 3 carbon atoms or alkoxy having 1 or 2 carbon atoms; particular preference is given to compounds of the formula Ie in which R(1) is COR(11) where R(11) is CₓH₂ₓ—R(14) where x is 1 and R(14) is phenyl, where phenyl is substituted by one substituent selected from the group consisting of alkoxy having 1 or 2 carbon atoms, for example methoxy.

In a further embodiment, preference is given to compounds of the formula Ie in which R(2) is hydrogen.

In a further embodiment, preference is given to compounds of the formula Ie in which R(3) is C_{y}H_{2y}—R(16) where y is 0, 1 or 2 and R(16) is alkyl having 1, 2 or 3 carbon atoms, cycloalkyl having 5 or 6 carbon atoms, phenyl or pyridyl, where phenyl and pyridyl are each unsubstituted or substituted by 1, 2 or 3 substituents selected from the group consisting of F, Cl, CF₃, alkyl having 1, 2 or 3 carbon atoms or alkoxy having 1 or 2 carbon atoms; particular preference is given to compounds of the formula Ie in which R(3) is C_{y}H_{2y}—R(16) where y is 1 and R(16) is phenyl, where phenyl is substituted by 2 substituents selected from the group consisting of F and Cl, for example F.

In a further embodiment, preference is given to compounds of the formula Ie in which R(4) is hydrogen.

In a further embodiment, preference is given to compounds of the formula Ie in which R(5), R(6) and R(7) are each hydrogen.

In a further embodiment, preference is given to compounds of the formula Ie in which R(30) and R(31) are each hydrogen.

In a further embodiment, preference is given to compounds of the formula If in which X is oxygen or sulfur.

In a further embodiment, preference is given to compounds of the formula If in which R(1) is C(O)OR(9) or COR(11), where R(9) and R(11) are each independently CₓH₂ₓ—R(14) where x is 0, 1, 2, 3 or 4 and R(14) is alkyl having 1, 2 or 3 carbon atoms, cycloalkyl having 5 or 6 carbon atoms or phenyl, where phenyl is unsubstituted or substituted by 1 or 2 substituents selected from the group consisting of F, Cl, Br, I, CF₃, OCF₃, OH, alkyl having 1, 2 or 3 carbon atoms or alkoxy having 1 or 2 carbon atoms; particular preference is given to compounds of the formula If in which R(1) is COR(11) where R(11) is CₓH₂ₓ—R(14) where x is 1 and R(14) is phenyl, where phenyl is substituted by one substituent selected from the group consisting of alkoxy having 1 or 2 carbon atoms, for example methoxy.

In a further embodiment, preference is given to compounds of the formula If in which R(2) is hydrogen.

In a further embodiment, preference is given to compounds of the formula If in which R(3) is C_{y}H_{2y}—R(16) where y is 0, 1, 2, 3 or 4 and R(16) is alkyl having 1, 2 or 3 carbon atoms, cycloalkyl having 5 or 6 carbon atoms, phenyl or pyridyl, where phenyl and pyridyl are each unsubstituted or substituted by 1, 2 or 3 substituents selected from the group consisting of F, Cl, CF₃, alkyl having 1, 2 or 3 carbon atoms or alkoxy having 1 or 2 carbon atoms; particular preference is given to compounds of the formula If in which R(3) is C_{y}H_{2y}—R(16) where y is 1 or 4 and R(16) is alkyl having 1, 2 or 3 carbon atoms, for example methyl, or phenyl, where phenyl is substituted by two substituents selected from the group consisting of F and Cl, for example F.

In a further embodiment, preference is given to compounds of the formula If in which R(4) is hydrogen.

In a further embodiment, preference is given to compounds of the formula If in which R(5), R(6) and R(7) are each hydrogen.

In a further embodiment, preference is given to compounds of the formula If in which R(30) and R(31) are each hydrogen.

In a further embodiment, preference is given to compounds of the formula Ih in which R(1) is

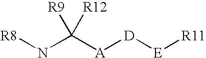

where A is —CₙH₂ₙ— where n is 0, 1 or 2, D is a bond or oxygen, E is —CₘH₂ₘ— where m is 0 or 1, R(8) is hydrogen or alkyl having 1, 2, 3 or 4 carbon atoms, R(9) is hydrogen or alkyl having 1, 2, 3 or 4 carbon atoms, R(12) is alkyl having 1, 2, 3 or 4 carbon atoms or cyclopropyl and R(11) is phenyl or pyridyl, where phenyl and pyridyl are each unsubstituted or substituted by 1, 2 or 3 substituents selected from the group consisting of F, Cl, CF₃, OCF₃, CN, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino; particular preference is given to compounds of the formula Ih in which R(1) is

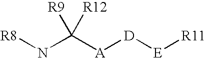

where A is —CₙH₂ₙ— where n is 0, D is a bond, E is —CₘH₂ₘ— where m is 0, R(8) and R(9) are each hydrogen, R(12) is alkyl having 1, 2 or 3 carbon atoms, for example ethyl, and R(11) is phenyl or pyridyl, where phenyl and pyridyl are each unsubstituted or substituted by one substituent selected from the group consisting of alkoxy having 1 or 2 carbon atoms, for example methoxy.

In a further embodiment, preference is given to compounds of the formula Ih in which R(2) is hydrogen.

In a further embodiment, preference is given to compounds of the formula Ih in which R(3) is alkyl having 3, 4 or 5 carbon atoms or phenyl, where phenyl is unsubstituted or substituted by 1, 2 or 3 substituents selected from the group consisting of F, Cl, CF₃, OCF₃, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino; particular preference is given to compounds of the formula Ih in which R(3) is alkyl having 3, 4 or 5 carbon atoms, for example 4 carbon atoms, or phenyl, where phenyl is substituted by one substituent selected from the group consisting of alkoxy having 1 or 2 carbon atoms, for example methoxy.

In a further embodiment, preference is given to compounds of the formula Ih in which R(5) is hydrogen or methoxy, and R(4), R(6) and R(7) are each hydrogen.

In a further embodiment, preference is given to compounds of the formula Ij in which R(1) is

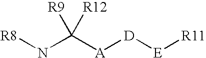

where A is —CₙH₂ₙ— where n is 0 or 1, D is a bond or —O—, E is —CₘH₂ₘ— where m is 0 or 1, R(8) is hydrogen or alkyl having 1, 2 or 3 carbon atoms, R(9) is hydrogen, ethyl or methyl, R(11) is phenyl, naphthyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl, where phenyl, naphthyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl are each unsubstituted or substituted by 1 or 2 substituents selected from the group consisting of F, Cl, CF₃, OCF₃, CN, COMe, methoxy, ethoxy, dimethylamino, sulfamoyl and methylsulfonyl, and R(12) is alkyl having 1, 2 or 3 carbon atoms, ethynyl, cyclopropyl, phenyl, naphthyl or heteroaryl, where phenyl, naphthyl and heteroaryl are each unsubstituted or substituted by 1 or 2 substituents selected from the group consisting of F, Cl, CF₃, OCF₃, CN, COMe, ethoxy, dimethylamino, sulfamoyl, methylsulfonyl and methylsulfonylamino; particular preference is given to compounds of the formula Ih in which R(1) is

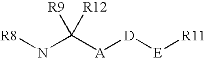

where A is —CₙH₂ₙ— where n is 0, D is a bond, E is —CₘH₂ₘ— where m is 0, R(8) is hydrogen, R(9) is hydrogen, R(11) is unsubstituted phenyl and R(12) is alkyl having 1, 2 or 3 carbon atoms, for example ethyl.

In a further embodiment, preference is given to compounds of the formula Ij in which R(2) is hydrogen.

In a further embodiment, preference is given to compounds of the formula Ij in which R(3) is heteroaryl, where heteroaryl is unsubstituted or substituted by 1 or 2 substituents selected from the group consisting of F, Cl, CF₃, OCF₃, CN, COMe, methyl, methoxy, ethoxy, dimethylamino, sulfamoyl and methylsulfonyl; particular preference is given to compounds of the formula Ih in which R(3) is furanyl, thienyl, pyrrolyl, imidazolyl, pyrazolyl, triazolyl, tetrazolyl, oxazolyl, isoxazolyl, thiazolyl, isothiazolyl, pyridyl, pyrazinyl, pyrimidinyl, pyridazinyl, indolyl, indazolyl, quinolyl, isoquinolyl, phthalazinyl, quinoxalinyl, quinazolinyl, cinnolinyl, for example quinolyl.

In a further embodiment, preference is given to compounds of the formula Ij in which R(5) is hydrogen or F, for example F, and R(4), R(6) and R(7) are each hydrogen. R(1) in the compounds of the formulae Ih and Ij is connected via the nitrogen atom in the residue R(1) to the carbonyl residue in the compounds of the formulae Ih and Ij.

Especially preferred is the use of compounds selected from the group of:
2-(Butyl-1-sulfonylamino)-N-[1(R)-(6-methoxypyridin-3-yl)propyl]benzamide,
2-(Butyl-1-sulfonylamino)-N-[1(S)-(6-methoxypyridin-3-yl)propyl]benzamide,
N-(2-Pyridin-3-ylethyl)-2′-{[2-(4-methoxyphenyl)acetylamino]methyl}biphenyl-2-carboxamide,
(S)-5-Fluoro-2-(quinoline-8-sulfonylamino-N-(1-phenylpropyl)benzamide,
(S)-5-Methoxy-2-(4-methoxybenzenesulfonylamino)-N-(1-phenylpropyl)benzamide,
N-(2-(R)-hydroxypropyl)-2′-(α-(S)-methylbenzyloxycarbonylaminomethyl)biphenyl-2-carboxamide,
N-(2,4-Difluorobenzyl)-5-chloro-2′-{[2-(4-methoxyphenyl)acetylamino]methyl}biphenyl-2-carboxamide,
Benzyl {2′-[methyl(2-pyridin-2-ylethyl)carbamoyl]biphenyl-2-ylmethylyl}carbamate
N-(2,4-Difluorobenzyl)-3-(2-{[2-(4-methoxyphenyl)acetylamino]methyl}-phenyl)thiophene-2-carboxamide,
N-(2,4-Difluorobenzyl)-5-(2-{[2-(4-methoxyphenyl)acetylamino]methyl}phenyl)furan-2-carboxamide,
N-(3-Methylbutyl)-2-(2-{[2-(4-methoxyphenyl)acetylamino]methyl}phenyl)furan-3-carboxamide,
N-(2,4-Difluorobenzyl)-2-(2-{[2-(4-methoxyphenyl)acetylamino]methyl}-phenyl)thiophene-3-carboxamide,
(S)-1-Phenylethyl {2-[2-(2-pyridin-2-ylethylcarbamoyl)pyridin-3-yl]benzyl}carbamate,
N-Cyclopropylmethyl-3-{2-[((R)-3-phenylbutyrylamino)methyl]phenyl}pyridine-2-carboxamide,
Benzyl {2-[3-(2,4-difluorobenzylcarbamoyl)pyridin-2-yl]benzyl}carbamate,
Benzyl {4-[3-(3-methylbutylcarbamoyl)phenyl]pyridin-3-ylmethyl}carbamate,
N-(3-Methylbutyl)-2′-{[3-(4-methoxyphenyl)propionylamino]methyl}-6-methylbiphenyl-3-carboxamide
and N-indan-1-yl-2-methyl-5-(3-methylbutylsulfamoyl)benzamide
and/or physiologically compatible salts thereof.

If in the compounds of the formulae I, Ia, Ib, Ic, Id, Ie, If, Ig, Ih and Ij any groups, substituents, ring members, numbers or other features such as, for example, R14, alkyl groups etc. occur several times, they can all independently of one another have any of the indicated meanings and can in each case be identical or different from one another.

Alkyl radicals and alkylene radicals may be straight-chain or branched. This also applies to the alkylene radicals of the formulae CᵣH₂ᵣ, CₓH₂ₓ, CₛH₂ₛ, C_{y}H_{2y}, C_{z}H_{2z}, CᵥH₂ᵥ, C_{w}H_{2w}, CₙH₂ₙ, CₘH₂ₘ, CₚH₂ₚ and (CH₂)ₓ. Alkyl radicals and alkylene radicals may also be straight-chain or branched when they are substituted or present in other radicals, for example in fluoroalkyl radicals or alkoxy radicals. Examples of alkyl radicals are methyl, ethyl, n-propyl, isopropyl (=1-methylethyl), n-butyl, isobutyl (=2-methylpropyl), sec-butyl (=1-methylpropyl), tert-butyl (=1,1-dimethylethyl), n-pentyl, isopentyl, tert-pentyl, neopentyl, hexyl and heptyl. The divalent radicals derived from these radicals, for example methylene, 1,1-ethylene, 1,2-ethylene, 1,1-propylene, 1,2-propylene, etc. are examples of alkylene radicals. Preferred alkyl radicals are methyl, ethyl, n-propyl, isopropyl, n-butyl, sec-butyl and tert.-butyl. In alkyl radicals, one or more, for example 1, 2, 3, 4, 5, 6, 7, 8, 9, 10, 11, 12, 13, 14 or 15, hydrogen atoms may be substituted by fluorine atoms. Examples of such fluoroalkyl radicals are trifluoromethyl, 2,2,2-trifluoroethyl and pentafluoroethyl. Substituted alkyl radicals may be substituted in any positions.

Alkynyl radicals may be straight-chain or branched. This is also the case when they bear substituents, for example in fluoroalkynyl radicals. The alkynyl radicals may be unsaturated in different positions and also be polyunsaturated. Examples of alkynyl radicals are ethynyl, n-prop-1-ynyl, n-prop-2-ynyl, n-but-1-ynyl, n-but-2-ynyl, n-but-3-ynyl, n-buta-1,3-diynyl and sec-but-2-ynyl (=1-methylprop-2-ynyl). In alkynyl radicals, one or more, for example 1, 2, 3, 4, 5, 6 or 7, hydrogen atoms may be substituted by fluorine atoms. Substituted alkynyl radicals may be substituted in any positions.

Examples of cycloalkyl radicals are cyclopropyl, cyclobutyl, cyclopentyl, cyclohexyl, cycloheptyl, cyclooctyl, cyclononyl, cyclodecanyl and cycloundecanyl. In cycloalkyl radicals, one or more, for example 1, 2, 3, 4, 5, 6, 7 or 8, hydrogen atoms may be substituted by fluorine atoms. Substituted cycloalkyl radicals may be substituted in any positions.

Aryl is, for example, phenyl and 2- or 3-naphthyl.

Phenyl radicals may be unsubstituted or mono- or polysubstituted, for example mono-, di- or trisubstituted, by identical or different radicals. When a phenyl radical is substituted, it preferably bears one or two identical or different substituents. This applies equally to substituted phenyl radicals in groups such as phenylalkyl or phenyloxy, for example. In monosubstituted phenyl radicals, the substituent may be present in the 2-position, the 3-position or the 4-position. Disubstituted phenyl may be substituted in the 2,3-position, 2,4-position, 2,5-position, 2,6-position, 3,4-position or 3,5-position. In trisubstituted phenyl radicals, the substituents may be present in the 2,3,4-position, 2,3,5-position, 2,4,5-position, 2,4,6-position, 2,3,6-position or 3,4,5-position.

Heteroaryl radicals are aromatic ring compounds in which one or more ring atoms are oxygen atoms, sulfur atoms or nitrogen atoms, for example 1, 2 or 3 nitrogen atoms, 1 or 2 oxygen atoms, 1 or 2 sulfur atoms or a combination of different heteroatoms. The heteroaryl radicals may be attached via all positions, for example via the 1-position, 2-position, 3-position, 4-position, 5-position, 6-position, 7-position or 8-position. Heteroaryl radicals may be unsubstituted or mono- or polysubstituted, for example mono-, di- or trisubstituted, by identical or different radicals. This applies equally to the heteroaryl radicals, as, for example, in the heteroarylalkyl radical. Heteroaryl is, for example, furanyl, thienyl, pyrrolyl, imidazolyl, pyrazolyl, triazolyl, tetrazolyl, oxazolyl, isoxazolyl, thiazolyl, isothiazolyl, pyridyl, pyrazinyl, pyrimidinyl, pyridazinyl, indolyl, indazolyl, quinolyl, isoquinolyl, phthalazinyl, quinoxalinyl, quinazolinyl and cinnolinyl. Heteroaryl radicals are in particular 2- or 3-thienyl, 2- or 3-furyl, 1-, 2- or 3-pyrrolyl, 1-, 2-, 4- or 5-imidazolyl, 1-, 3-, 4- or 5-pyrazolyl, 1,2,3-triazol-1-, -4- or -5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4- or -5-yl, 1,2,4-oxadiazol-3- or -5-yl, 1,3,5-oxadiazol-2-yl or -5-yl, 2-, 4- or 5-thiazolyl, 3-, 4- or 5-isothiazolyl, 1,3,4-thiadiazol-2- or -5-yl, 1,2,4-thiadiazol-3- or -5-yl, 1,2,3-thiadiazol-4- or -5-yl, 2-, 3- or 4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-quinolyl, 1-, 3-, 4-, 5-, 6-, 7- or 8-isoquinolyl, 2-, 4-, 5-, 6-, 7- or 8-quinazolinyl, 3-, 4-, 5-, 6-, 7- or 8-cinnolinyl, 2-, 3-, 5-, 6-, 7- or 8-quinoxalinyl, 1-, 4-, 5-, 6-, 7- or 8-phthalazinyl. Also included are the corresponding N-oxides of these compounds, i.e., for example, 1-oxy-2-, -3- or -4-pyridyl.

Particular preference is given to the heteroaromatics 2- or 3-thienyl, 2- or 3-furyl, 1-, 2- or 3-pyrrolyl, 1-, 2-, 4- or 5-imidazolyl, 2-, 3-, 4-, 5-, 6-, 7- or 8-quinolyl, 1-, 3-, 4- or 5-pyrazolyl, 2-, 3- or 4-pyridyl, 2- or 3-pyrazinyl, 2-, 4-, 5- or 6-pyrimidinyl and 3- or 4-pyridazinyl.

N-containing heterocycles are ring compounds in which one or more ring atoms are nitrogen atoms, for example 1, 2 or 3 nitrogen atoms. The N-containing heterocycles may be attached via all positions, for example via the 1-position, 2-position, 3-position, 4-position, 5-position, 6-position, 7-position or 8-position. N-containing heterocycles may be unsubstituted or mono- or polysubstituted, for example mono-, di- or trisubstituted, by identical or different radicals. The N-containing heterocycles having 1, 2, 3, 4, 5, 6, 7, 8 or 9 carbon atoms are in particular the aromatic systems 1-, 2- or 3-pyrrolyl, 1-, 2-, 4- or 5-imidazolyl, 1-, 3-, 4- or 5-pyrazolyl, 1,2,3-triazol-1-, -4- or -5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4- or -5-yl, 1,2,4-oxadiazol-3- or -5-yl, 1,3,4-oxadiazol-2-yl or -5-yl, 2-, 4- or 5-thiazolyl, 3-, 4- or 5-isothiazolyl, 1,3,4-thiadiazol-2- or -5-yl, 1,2,4-thiadiazol-3- or -5-yl, 1,2,3-thiadiazol-4- or -5-yl, 2-, 3- or -4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-quinolyl, 1-, 3-, 4-, 5-, 6-, 7- or 8-isoquinolyl, 2-, 4-, 5-, 6-, 7- or 8-quinazolinyl, 3-, 4-, 5-, 6-, 7- or 8-cinnolinyl, 2-, 3-, 5-, 6-, 7- or 8-quinoxalinyl, 1-, 4-, 5-, 6-, 7- or 8-phthalazinyl.

Particular preference is given to the N-containing heterocycles pyrrolyl, imidazolyl, quinolyl, pyrazolyl, pyridyl, pyrazinyl, pyrimidinyl and pyridazinyl.

N-containing heteroaromatics are aromatic ring compounds in which one or more ring atoms are nitrogen atoms, for example 1, 2 or 3 nitrogen atoms. The N-containing heteroaromatics may be attached via all positions, for example via the 1-position, 2-position, 3-position, 4-position, 5-position, 6-position, 7-position or 8-position. N-containing heteroaromatics may be unsubstituted or mono- or polysubstituted, for example mono-, di- or trisubstituted, by identical or different radicals. The N-containing heteroaromatics having 1, 2, 3, 4, 5, 6, 7, 8 or 9 carbon atoms are in particular the aromatic systems 1-, 2- or 3-pyrrolyl, 1-, 2-, 4- or 5-imidazolyl, 1-, 3-, 4- or 5-pyrazolyl, 1,2,3-triazol-1-, -4- or -5-yl, 1,2,4-triazol-1-, -3- or -5-yl, 1- or 5-tetrazolyl, 2-, 4- or 5-oxazolyl, 3-, 4- or 5-isoxazolyl, 1,2,3-oxadiazol-4- or -5-yl, 1,2,4-oxadiazol-3- or -5-yl, 1,3,4-oxadiazol-2-yl or -5-yl, 2-, 4- or 5-thiazolyl, 3-, 4- or 5-isothiazolyl, 1,3,4-thiadiazol-2- or -5-yl, 1,2,4-thiadiazol-3- or -5-yl, 1,2,3-thiadiazol-4- or -5-yl, 2-, 3- or -4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-quinolyl, 1-, 3-, 4-, 5-, 6-, 7- or 8-isoquinolyl, 2-, 4-, 5-, 6-, 7- or 8-quinazolinyl, 3-, 4-, 5-, 6-, 7- or 8-cinnolinyl, 2-, 3-, 5-, 6-, 7- or 8-quinoxalinyl, 1-, 4-, 5-, 6-, 7- or 8-phthalazinyl. Also included are the corresponding N-oxides of these compounds, i.e., for example, 1-oxy-2-, -3- or -4-pyridyl.

Particular preference is given to the N-containing heterocycles pyrrolyl, imidazolyl, quinolyl, pyrazolyl, pyridyl, pyrazinyl, pyrimidinyl and pyridazinyl.

Pyridyl is 2-, 3- or 4-pyridyl. Thienyl is 2- or 3-thienyl. Furyl is 2- or 3-furyl. In the case of di- or polysubstitution of a radical, the substituents may be the same or different.

When the compounds of the formula Ia, Ib, Ic, Id, Ie, If, Ig, Ih or Ij contain one or more acidic or basic groups or one or more basic heterocycles, the corresponding physiologically or toxicologically compatible salts are also included in the invention, especially the pharmaceutically usable salts. For instance, the compounds of the formula Ia, Ib, Ic, Id, Ie, If, Ig, Ih or Ij may be deprotonated on the sulfonamide group and be used, for example, as alkali metal salts, preferably sodium or potassium salts, or as ammonium salts, for example as salts with ammonia or organic amines or amino acids. Compounds of the formula Ia or Ib which contain a pyridine or quinoline substituent may also be used in the form of their physiologically compatible acid addition salts with inorganic or organic acids, for example as hydrochlorides, phosphates, sulfates, methanesulfonates, acetates, lactates, maleates, fumarates, malates, gluconates, etc. The compounds of the formula Ia or Ib may also be present as trifluoroacetates.

The compounds of the formula Ia, Ib, Ic, Id, Ie, If, Ig, Ih or Ij may be present in stereoisomeric forms in the case of appropriate substitution. When the compounds of the formula Ia, Ib, Ic, Id, Ie, If, Ig, Ih or Ij contain one or more centers of asymmetry, these may each independently have S-configuration or R-configuration. The invention includes all possible stereoisomers, for example enantiomers or diastereomers, and mixtures of two or more stereoisomeric forms, for example enantiomers and/or diastereomers, in any ratios. Enantiomers, for example, are thus included in the invention in enantiomerically pure form, both as levorotatory and as dextrorotatory antipodes, and also in the form of mixtures of the two enantiomers in different ratios or in the form of racemates. The preparation of individual stereoisomers can, if desired, be effected by separating a mixture by customary methods or, for example, by use of isomerically pure synthesis units.

The present invention encompasses all tautomeric forms of the compounds of the formula Ia, Ib, Ic, Id, Ie, If, Ig, Ih or Ij.

The compounds of the formulae Ia, Ib, Ic, Id, Ie, If, Ig, Ih or Ij can be prepared in accordance with the preparation methods which are described in WO01/00573, WO1/025189, WO02/044137, WO02/046162, WO02/048131, WO02/087568, WO02/088073, WO02/100825.

The compounds of the formulae Ia, Ib, Ic, Id, Ie, If, Ig, Ih or Ij can be used alone, in a mixture with one another or in the form of pharmaceutical formulations on humans or animals, in accordance with the invention, for the therapy or prophylaxis of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer.

Pharmaceutical formulations comprise, as an active constituent, an effective dose of at least one compound of the formula Ia, Ib, Ic, Id, Ie, If, Ig, Ih and/or Ij and/or of a physiologically compatible salt thereof in addition to customary, pharmaceutically unobjectionable carriers and assistants and optionally also one or more other active pharmacological ingredients. The pharmaceutical formulations contain normally from 0.1 to 90% by weight of the compounds of the formulae Ia to Ij and/or physiologically compatible salts thereof.

The pharmaceutical formulations can be produced in a manner known per se. To this end, the active ingredients and/or their physiologically compatible salts, together with one or more solid or liquid pharmaceutical carriers and/or assistants, are converted to a suitable administration form or dosage form, which can then be used as a medicament in human medicine or veterinary medicine.

Medicaments which comprise inventive compounds of the formulae Ia to Ij and/or their pharmaceutically compatible salts can be administered, for example, orally, parenterally, intravenously, rectally, nasally, by inhalation or topically, especially orally, intravenously or nasally, the preferred administration depending on the particular case.

The excipients which are suitable for the desired pharmaceutical formulation are familiar to those skilled in the art on the basis of their expert knowledge. In addition to solvents, gel formers, suppository bases, tablet excipients and other active ingredient carriers, it is possible to use, for example, antioxidants, dispersants, emulsifiers, antifoams, flavorings, preservatives, solubilizers, agents for achieving a depot effect, buffer substances or colorings.

For an oral administration form, the active compounds are mixed with the additives suitable for this purpose, such as carriers, stabilizers or inert diluents and converted to the suitable dosage forms, such as tablets, coated tablets, hard gelatin capsules, aqueous, alcoholic or oily solutions, by the customary methods. Examples of useful inert carriers include gum arabic, magnesia, magnesium carbonate, potassium phosphate, lactose, glucose or starch, in particular corn starch. The preparation may be either in the form of dry granules or in the form of moist granules. Examples of useful oily carriers or useful solvents are vegetable or animal oils, such as sunflower oil or cod liver oil. Useful solvents for aqueous or alcoholic solutions include, for example, water, ethanol or sugar solutions or mixtures thereof. Further assistants, also for other administration forms, are, for example, polyethylene glycols and polypropylene glycols.

For subcutaneous, intramuscular or intravenous administration, the active compounds used, if desired with the substances customary for this purpose, such as solubilizers, emulsifiers or further excipients, are converted to solution, suspension or emulsion.

Examples of useful solvents are: water, physiological saline or alcohols, for example ethanol, propanol, glycerol, and additionally also sugar solutions such as glucose or mannitol solutions, or else a mixture of the different solvents mentioned. Examples of suitable pharmaceutical formulations for administration in the form of aerosols or sprays, for example for nasal administration, are solutions, suspensions, emulsions or vesicular and micellar medicament forms of the active ingredients or their physiologically compatible salts in water or in a pharmaceutically unobjectionable water-miscible or oily solvent, or a mixture of such solvents. Also suitable for administration in the form of aerosols or sprays, for example for nasal administration, are powders of the active ingredients or their physiologically compatible salts. If required, all formulations may also comprise other pharmaceutical excipients such as isotonizing additives, surfactants, emulsifiers and stabilizers, and also a propellant gas. The formulations mentioned may additionally be in the form of freeze-dried products. The preparations contain the active ingredient typically in a concentration of from about 0.001 to 10% by weight, in particular from about 0.05 to 5% by weight.

The dosage of the active compounds or of the physiologically compatible salts thereof to be administered in accordance with the invention depends upon the individual case and, for optimal action, should be adjusted to the circumstances of the individual case as usual. For instance, it depends of course upon the frequency of administration and upon the potency and duration of action of the compounds used in each case for therapy or prophylaxis, but also upon the nature and severity of the disease to be treated, and also on the gender, age, weight and individual responsiveness of the human or animal to be treated, and upon whether acute or chronic therapy or prophylaxis is being practiced.

The dosage of the compounds of the formulae Ia, Ib, Ic, Id, Ie, If, Ig, Ih and/or Ij may typically vary within the range from 1 mg to 1 g per day and per person (at body weight about 75 kg), preferably from 5 to 750 mg per day and person. However, higher doses may also be appropriate. The daily dose of the active ingredients may be administered all at once or it may be divided between a plurality of, for example 2, 3 or 4, administrations.

### EXPERIMENTAL PART

### List of Abbreviations

| | |
|---|---|
| EDAC | N-ethyl-N′-(3-dimethylaminopropyl)carbodiimide hydrochloride |
| EMG | electromyographical |
| DMSO | dimethyl sulfoxide |
| HOBT | 1-hydroxy-1H-benzotriazole |
| n.s. | not significant |
| PEG | polyethylene glycol |
| THF | tetrahydrofuran |
| SEM | standard error |
| vs. | in comparison to (versus) |

### Example 1

### 2-(Butyl-1-sulfonylamino)-N-[1(R)-(6-methoxypyridin-3-yl)propyl]benzamide and 2-(butyl-1-sulfonylamino)-N-[1(S)-(6-methoxypyridin-3-yl)propyl]benzamide

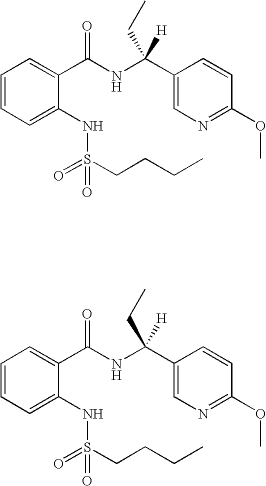

### a) 2-(Butyl-1-sulfonylamino)benzoic acid

20 g (188 mmol) of sodium carbonate were added to a suspension of 20 g (146 mmol) of 2-aminobenzoic acid in 250 ml of water. Subsequently, 11.4 g (72.8 mmol) of butylsulfonyl chloride were added dropwise and the reaction mixture was stirred at room temperature for 2 days. The mixture was acidified with concentrated hydrochloric acid and stirred at room temperature for 3 hours, and the precipitated product was filtered off with suction. Drying under reduced pressure afforded 9.6 g of 2-(butyl-1-sulfonylamino)benzoic acid.

### b) 1-(6-Methoxypyridin-3-yl)propylamine

3 ml (23.2 mmol) of 5-bromo-2-methoxypyridine were added at −70° C. to a solution of 10.2 ml of butyllithium (2.5 M solution in hexane; 25.5 mmol) in 50 ml of diethyl ether. After 10 min, 1.4 ml (19.5 mmol) of propionitrile were added. After 2 hours at −70° C., the reaction mixture was allowed to come slowly to room temperature. 2.2 g of sodium sulfate decahydrate were then added and the mixture was left to stir for 1 hour. After subsequently adding 5 g of magnesium sulfate and stirring briefly, the salts were filtered off and the filtrate was concentrated. The residue was dissolved in 70 ml of methanol and, at 0° C., 1.1 g (28 mmol) of sodium borohydride were added. After stirring overnight, the reaction mixture was adjusted to pH 2 with concentrated hydrochloric acid and concentrated on a rotary evaporator. A residue was admixed with 10 ml of water and extracted once with diethyl ether. Subsequently, the aqueous phase was extracted with sodium hydrogencarbonate and concentrated under reduced pressure, and the residue was extracted with ethyl acetate. Drying and concentration of the ethyl acetate extracts afforded 1.4 g of racemic 1-(6-methoxypyridin-3-yl)propylamine.

### c) 2-(Butyl-2-sulfonylamino)-N-[1(R)-(6-methoxypyridin-3-yl)propyl]benzamide

4.4 g (32.7 mmol) of 1-hydroxy-1H-benzotriazole and 6.3 g (32.7 mmol) of N-ethyl-N′-(3-dimethylaminopropyl)carbodiimide hydrochloride were added to a solution of 8.0 g (31.1 mmol) of 2-(butyl-1-sulfonylamino)benzoic acid in 250 ml of tetrahydrofuran, and the reaction mixture was stirred for 90 min. A solution of 5.4 g (32.7 mmol) of racemic 1-(6-methoxypyridin-3-yl)propylamine in 20 ml of tetrahydrofuran was then added dropwise and the mixture was stirred overnight. The reaction mixture was admixed with 250 ml of water and extracted with 300 ml of ethyl acetate. The organic phase was extracted 5 times with 100 ml each time of saturated sodium hydrogencarbonate solution and then dried over magnesium sulfate. This afforded 9.0 g of 2-(butyl-1-sulfonylamino)-N-[1-(6-methoxypyridin-3-yl)propyl]benzamide.

The enantiomers were separated by preparative HPLC on a Chiralpak ADH column (250×4.6 mm); eluent: heptane/ethanol/methanol 10:1:1; temperature: 30° C.; flow rate: 1 ml/min. 4.0 g of 2-(butyl-1-sulfonylamino)-N-[1(R)-(6-methoxypyridin-3-yl)propyl]benzamide were eluted first at a retention time of 5.9 min. After a mixed fraction, 3.0 g of 2-(butyl-1-sulfonylamino)-N-[1(S)-(6-methoxypyridin-3-yl)propyl]benzamide were obtained at a retention time of 7.2 min.

2 g of the 2-(butyl-1-sulfonylamino)-N-[1(R)-(6-methoxypyridin-3-yl)propyl]benzamide were dissolved with heating in 9 ml of isopropanol, then 8 ml of warm water were added and the reaction mixture was allowed to cool slowly overnight. Filtration with suction at 0° C. afforded 1.5 g of 2-(butyl-1-sulfonylamino)-N-[1(R)-(6-methoxypyridin-3-yl)propyl]benzamide as colorless needle-like crystals; melting point 97° C.

### Example 2

### N-(2-pyridin-3-ylethyl)-2′-{[2-(4-methoxyphenyl)acetylamino]-methyl}biphenyl-2-carboxamide

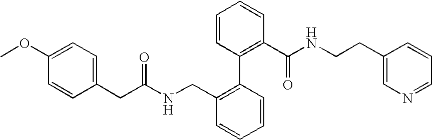

15.5 g (0.115 mol) of HOBT and 21.9 g (0.115 mol) of EDAC were added to a solution of 37.8 g (0.11 mol) of 2′-(tert-butoxycarbonylaminomethyl)biphenyl-2-carboxylic acid (Brandmeier, V.; Sauer, W. H. B.; Feigel, M.; Helv. Chim. Acta 1994, 77(1), 70-85) in 550 ml of THF, and the reaction mixture was stirred at room temperature for 45 min. Subsequently, 14.0 g (0.115 mol) of 3-(2-aminoethyl)pyridine were added and the mixture was stirred at RT overnight. After addition of 400 ml of water and 500 ml of ethyl acetate and intensive stirring, the phases were separated. The organic phase was washed once with 400 ml of saturated sodium chloride solution and twice with 400 ml each time of saturated sodium hydrogencarbonate solution. After drying over magnesium sulfate in the presence of activated carbon, the mixture was filtered and concentrated on a rotary evaporator.

The resulting intermediate (40.7 g) was dissolved in 600 ml of methylene chloride and then 100 ml of trifluoroacetic acid were slowly added dropwise. After stirring overnight, the reaction mixture was concentrated under reduced pressure. The residue was admixed with 250 ml of ethyl acetate and concentrated again in order to distill out excess trifluoroacetic acid. 72.8 ml (530 mmol) of triethylamine were added dropwise to the resulting crude product dissolved in 170 ml of methylene chloride, and 1 g of DMAP were added. Subsequently, at 5-10° C., 18.7 g (100 mmol) of 4-methoxyphenylacetyl chloride were added dropwise within 30 min, and the mixture was stirred at room temperature overnight. After addition of 150 ml of water and intensive stirring, the phases were separated and the organic phase was washed once with 100 ml of sodium chloride solution, once with 25 ml of 1M hydrochloric acid and twice with 100 ml each time of saturated sodium hydrogencarbonate solution. After drying over magnesium sulfate and activated carbon, the mixture was concentrated under reduced pressure. The resulting oil was dissolved in hot acetonitrile and allowed to crystallize out slowly. 21.5 g of N-(2-pyridin-3-ylethyl)-2′-{[2-(4-methoxyphenyl)acetylamino]methyl}biphenyl-2-carboxamide, melting point 116° C., were obtained.

### Example 3

### (S)-5-Fluoro-2-(quinoline-8-sulfonylamino)-N-(1-phenylpropyl)benzamide sodium salt

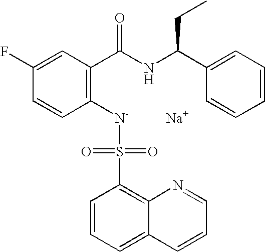

### a) 5-Fluoro-2-(quinoline-8-sulfonylamino)benzoic acid

A reaction mixture of 10.0 g (64 mmol) of 5-fluoro-2-aminobenzoic acid, 16.3 g (193 mmol) of sodium hydrogencarbonate and 16.3 g of 8-quinolinesulfonyl chloride in 325 ml of water and 325 ml of ethyl acetate was stirred at room temperature overnight. The aqueous phase was removed and extracted once with 50 ml of ethyl acetate. Subsequently, the aqueous phase was acidified with conc. hydrochloric acid and stirred for 2 h. The precipitated solid was filtered off with suction and dried under reduced pressure to obtain 19.5 g of 5-fluoro-2-(quinoline-8-sulfonylamino)benzoic acid.

### b) (S)-5-Fluoro-2-(quinoline-8-sulfonylamino)-N-(1-phenylpropyl)benzamide

5.5 g (15.9 mmol) of 5-fluoro-2-(quinoline-8-sulfonylamino)benzoic acid and 2.3 g (16.7 mmol) of (S)-phenylpropylamine were used to obtain 5.7 g of the title compound according to the method in WO 02100825.

m.p.: 163° C. P c) 2 ml of a 30% sodium methoxide solution were added to a solution of 5 g of (S)-5-fluoro-2-(quinoline-8-sulfonylamino)-N-(1-phenylpropyl)benzamide in 120 ml of ethyl acetate. The precipitated sodium salt was filtered off with suction and recrystallized from 25 ml of ethanol to obtain 3.3 g of (S)-5-fluoro-2-(quinoline-8-sulfonylamino)-N-(1-phenylpropyl)benzamide sodium salt.

### Example 4

### (S)-5-Methoxy-2-(4-methoxybenzenesulfonylamino)-N-(1-phenylpropyl)benzamide

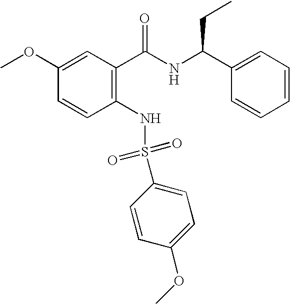

The compound was obtained according to the synthesis method specified in WO02088073.

### Example 5

### N-(2-(R)-hydroxypropyl)-2′-(α-(S)-methylbenzyloxycarbonyl-aminomethyl)biphenyl-2-carboxamide

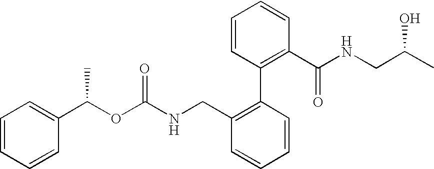

The compound was obtained according to the synthesis method specified in WO0125189.

### Example 6

### N-(2,4-Difluorobenzyl)-5-chloro-2′-{[2-(4-methoxyphenyl)acetyl-amino]methyl}biphenyl-2-carboxamide

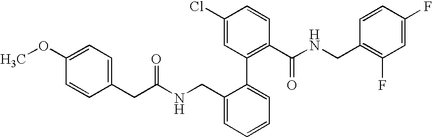

The compound was obtained according to the synthesis method specified in WO0125189.

### Example 7

### Benzyl {2′-[methyl-(2-pyridin-2-ylethyl)carbamoyl]biphenyl-2-ylmethyl}carbamate

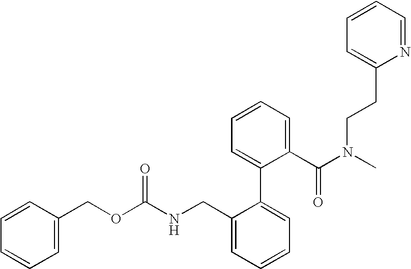

The compound was obtained according to the synthesis method specified in WO0125189.

### Example 8

### N-(2,4-Difluorobenzyl)-3-(2-{[2-(4-methoxyphenyl)acetyl-amino]methyl}phenyl)thiophene-2-carboxamide

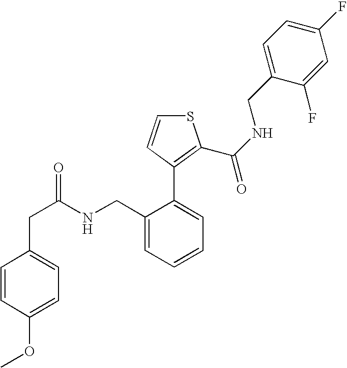

The compound was obtained according to the synthesis method specified in WO0248131.

### Example 9

### N-(2,4-Difluorobenzyl)-5-(2-{[2-(4-methoxyphenyl)acetyl-amino]methyl}phenyl)furan-2-carboxamide

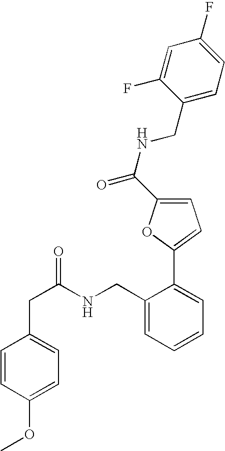

The compound was obtained according to the synthesis method specified in WO0248131.

### Example 10

### N-(3-methylbutyl)-2-(2-{[2-(4-methoxyphenyl)acetyl-amino]methyl}phenyl)furan-3-carboxamide

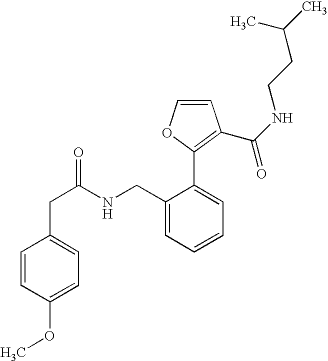

The compound was obtained according to the synthesis method specified in WO0248131.

### Example 11

### N-(2,4-Difluorobenzyl)-2-(2-{[2-(4-methoxyphenyl)acetyl-amino]methyl}phenyl)thiophene-3-carboxamide

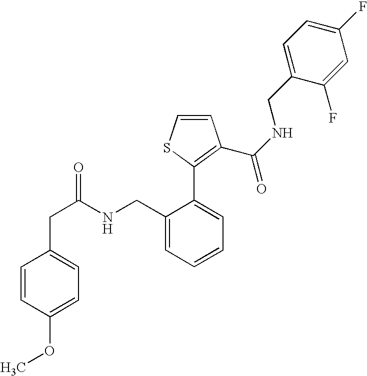

The compound was obtained according to the synthesis method specified in WO0248131.

### Example 12

### (S)-1-Phenylethyl {2-[2-(2-pyridin-2-ylethylcarbamoyl)pyridin-3-yl]benzyl}carbamate

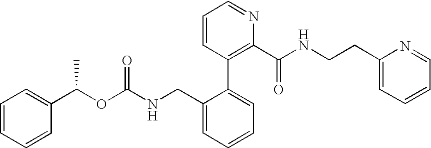

The compound was obtained according to the synthesis method specified in WO 0246162.

### Example 13

### N-Cyclopropylmethyl-3-{2-[((R)-3-phenylbutyrylamino)methyl]-phenyl}pyridine-2-carboxamide

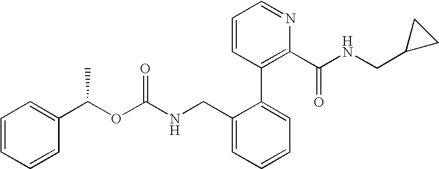

The compound was obtained according to the synthesis method specified in WO0246162.

### Example 14

### Benzyl {2-[3-(2,4-Difluorobenzylcarbamoyl)pyridin-2-yl]benzyl}carbamate

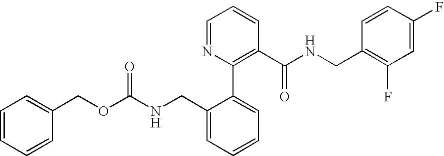

The compound was obtained according to the synthesis method specified in WO0246162.

### Example 15

### Benzyl {4-[3-(3-methylbutylcarbamoyl)phenyl]pyridin-3-yl-methyl}carbamate

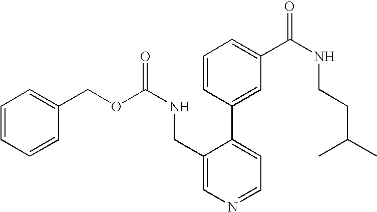

The compound was obtained according to the synthesis method specified in WO0246162.

### Example 16

### N-(3-Methylbutyl)-2′-{[3-(4-methoxyphenyl)propionylamino]methyl}-6-methylbiphenyl-3-carboxamide

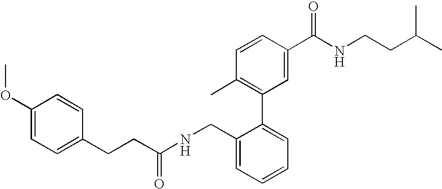

The compound was obtained according to the synthesis method specified in WO0244137.

### Example 17

### N-Indan-1-yl-2-methyl-5-(3-methylbutylsulfamoyl)benzamide

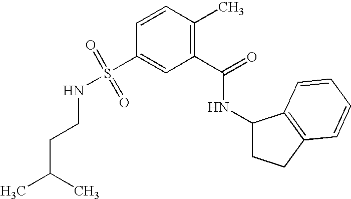

The compound was obtained according to the synthesis method specified in WO0100573.

### Pharmacological Investigations

### A) Determination of the Activity on the TASK-1 Channel in Xenopus Oocytes

Mouse or human TASK-1 channels were expressed in Xenopus oocytes. For this purpose, oocytes were first isolated from Xenopus levis and defolliculated. Subsequently, TASK-1-encoding RNA synthesized in vitro was injected into these oocytes. After two days of TASK-1 protein expression, TASK-1 currents were measured on the oocytes with the two-microelectrode voltage clamp technique. In this measurement, the TASK-1 channels were generally activated with voltage jumps lasting 250 ms to 40 mV. The bath was flushed with a solution of the following composition: NaCl 96 mM, KCl 2 mM, CaCl₂ 1.8 mM, MgCl₂ 1 mM, HEPES 5 mM (titrated to pH 7.4 with NaOH). These experiments were performed at room temperature. For data acquisition and analysis, the following were used: Geneclamp amplifier (Axon Instruments, Foster City, USA) and MacLab D/A converter and software (ADinstruments, Castle Hill, Australia). The inventive substances were tested by adding them to the bath solution in different concentrations. The effects of the substances were calculated as the percentage inhibition of the TASK-1 control current which was obtained when no substance was added to the solution. The data was subsequently fitted to the Hill equation in order to determine the half-maximum inhibitory concentrations (IC₅₀ values) for the particular substances.

In this way, the following IC₅₀ values were determined for the compounds listed below:

| | IC₅₀ (μmol) | IC₅₀ (μmol) |
|---|---|---|
| Compound | TASK1 (mouse) | TASK1 (human) |
| Example 1 | 0.15 | 0.10 |
| (R-enantiomer) | | |
| Example 1 | 0.80 | |
| (S-enantiomer) | | |
| Example 2 | 0.43 | 0.57 |
| Example 3 | 0.82 | |
| Example 4 | 0.65 | |
| Example 5 | 2.08 | |
| Example 6 | 1.15 | |
| Example 7 | 1.06 | |
| Example 8 | 0.55 | |
| Example 9 | 2.37 | |
| Example 10 | 1.19 | |
| Example 11 | 0.35 | |
| Example 12 | 3.36 | |
| Example 13 | 3.07 | |
| Example 14 | 2.90 | |
| Example 15 | 1.83 | |
| Example 16 | 2.77 | |

B) Determination of the Activity on the TASK-1 Channel with the FLIPR Technique

With the aid of an FLIPR assay, the inhibition of the human TASK-1 current by the example compounds was measured at a concentration of 10 μmol/l on CHO cells in which the human TASK-1 channel is expressed. This gave the following inhibition values:

| Compound | Inhibition at 10 μmol/l |
|---|---|
| Example 1 | 81% |
| (R-enantiomer) | |
| Example 1 | 58% |
| (S-enantiomer) | |
| Example 2 | 77% |
| Example 3 | 82% |
| Example 5 | 88% |
| Example 6 | 68% |
| Example 7 | 57% |
| Example 8 | 77% |
| Example 9 | 60% |
| Example 10 | 61% |
| Example 11 | 88% |
| Example 12 | 73% |
| Example 13 | 65% |
| Example 14 | 50% |
| Example 15 | 70% |

### C) Determination of the Activity on the TASK-3 Channel by Patch-Clamp Investigations

With the aid of the patch-clamp technique, the inhibition of the human TASK-3 current by the example compounds was measured on CHO cells in which the human TASK-3 channel is expressed. To produce the TASK-3 cell line, the human TASK-3 cDNA (Genbank, Accession Number AF248241) was cloned into the eukaryotic expression vector p658, which bears a DHFR (dihydrofolate reductase) resistance gene [reference: Gene 1994 (149), 341-344, 1994]. CHO (Chinese hamster ovary) DHFR-minus cells were transfected with the TASK-3 expression construct using the Fugene reagent (Roche Biochemicals) according to the manufacturer's instructions. Recombinant DHFR-positive cells were cultivated in MEM (minimal essential medium) with addition of 10% dialyzed calf serum. Resulting cell clones were analyzed for the expression of TASK-3 with the aid of a fluorescence-based activity assay in a FlexStation (Molecular Devices) and with a membrane potention-sensitive dye (Molecular Devices FMP Dye Kit). Functional expression of TASK-3 was demonstrated by the increase in the fluorescence signal after addition of 50 mM KCl to the cells, which corresponds to a depolarization of the membrane potential. TASK-3-positive cell clones were subsequently analyzed for resulting potassium currents with the patch-clamp technique. The cell clone CHO-244-8-1 was selected as the representative cell clone for subsequent investigations.

To investigate the substances, the cells were introduced into a measurement chamber which is mounted on an inverted microscope. A micropipette drawn from borosilicate glass was pressed cautiously onto a cell with visual observation. Gentle suction establishes a high-resistance seal between glass pipette and cell. Brief suction tore open the membrane patch and established a whole-cell leakage. By applying voltage jumps of −140 mV to +80 mV, the electrical current was registered under voltage clamp conditions with the aid of an electronic patch-clamp amplifier (Axopatch-1D). The action of a substance was registered by addition in rising concentrations into the bath solution. The concentration of the half-maximum inhibition of the current (IC₅₀ value) was determined by fitting the curve to the mathematical Hill equation.

In this analysis, the following IC₅₀ values were determined:

| | IC₅₀ (μmol/l) |
|---|---|
| Compound | TASK3 (human) |
| Example 1 | 1.0 μM |
| (R-enantiomer) | |

### D) Investigation of the Action on Respiration in Rabbits

An apnea was induced in the rabbit by infusion of the narcotic propofol, 10 mg/kg/min. The vehicle used for the compound of example 1 (R-enantiomer) was DMSO/PEG (0.2 ml/1.8 ml). The time from the start of propofol infusion up to the apnea was recorded. In the control, the apnea set in after approx. 2.92 min; after administration of 10 mg/kg i.v. of the compound of example 1 (R-enantiomer), the onset of the apnea was delayed and did not set in until 5.63 min after commencement of the profopol infusion (table 1).

TABLE 1
(p < 0.001; number n = 13):

| | | | Example 1 |
|---|---|---|---|
| | Basal | Vehicle | (R-enantiomer) |
| Mean | 2.87 min | 2.92 min | 5.63 min |
| Standard deviation | 0.69 min | 0.60 min | 2.08 min |

The investigations demonstrate the effect of example 1 against central apneas.

### E) Investigation of the Effect on the Electromyographic Activity of the Genioglossus Muscle and the Respiratory Minute Volume in Rats

The compound of example 1 (R-enantiomer) and of example 2 was investigated for electromyographic activity of the genioglossus muscle and for respiration-stimulating effect on male urethane-chloralose-narcotized, vagotomized rats with a weight of from 250 to 300 g. For this purpose, the genioglossus EMG activity was measured by means of EMG electrodes. The respiration-stimulating effect was investigated by measuring the respiratory minute volume by means of a tracheal cannula. For the investigations, the rats were administered intravenously successively with 1, 3 and 10 mg/kg of the compound of example 1 (R-enantiomer) and of example 2, with glycofurol (50%) as the vehicle, at 15 minute intervals. CO₂ was used as the positive control for the stimulation of the genioglossus activity and of the respiratory minute volume.

TABLE 2
Effect of example 1 (R-enantiomer) on the EMG activity of the genioglossus
muscle in narcotized rats (number n = 8). EMG activity in arbitrary units

| | | | | | | Example 1 (R-enantiomer) | | |
|---|---|---|---|---|---|---|---|---|
| EMG activity | basal | 5% CO₂ | basal | vehicle | basal | 1 mg/kg | 3 mg/kg | 10 mg/kg |
| Mean | 3.83 | 5.13 | 3.66 | 3.85 | 3.52 | 4.45 | 5.59 | 7.18 |
| SEM | 0.48 | 0.64 | 0.42 | 0.46 | 0.35 | 0.44 | 0.57 | 0.49 |
| p < vs. vehicle | | 0.0001 | | | | 0.001 | 0.0001 | 0.0001 |

TABLE 3
Effect of example 1 (R-enantiomer) on respiration (respiratory minute volume
in ml/min) on narcotized rats (number n = 8)

| | | | | | | Example 1 (R-enantiomer) | | |
|---|---|---|---|---|---|---|---|---|
| EMG activity | basal | 5% CO₂ | basal | vehicle | basal | 1 mg/kg | 3 mg/kg | 10 mg/kg |
| Mean | 158 | 237 | 157 | 204 | 168 | 246 | 317 | 375 |
| SEM | 12 | 22 | 9 | 20 | 11 | 21 | 31 | 25 |
| p < vs. vehicle | | 0.0257 | | | | n.s. | 0.0306 | <.0001 |

TABLE 4
Effect of example 2 on the EMG activity of the genioglossus muscle in
narcotized rats (number n = 11). EMG activity in arbitrary units

| | | | | | | Example 2 | | |
|---|---|---|---|---|---|---|---|---|
| EMG | | 5% | | vehi- | | | | 10 |
| activity | basal | CO₂ | Basal | cle | basal | 1 mg/kg | 3 mg/kg | mg/kg |
| Mean | 2.37 | 3.15 | 2.31 | 2.46 | 2.17 | 3.15 | 3.55 | 3.82 |
| SEM | 0.39 | 0.51 | 0.41 | 0.42 | 0.39 | 0.54 | 0.59 | 0.63 |
| p < | | 0.001 | | | | 0.001 | 0.001 | 0.001 |
| vs. | | | | | | | | |
| vehicle | | | | | | | | |

TABLE 5
Effect of example 2 on the respiration (respiratory minute volum
in ml/min) on narcotized rats (number n = 11)

| | | | | | | Example 2 | | |
|---|---|---|---|---|---|---|---|---|
| EMG | | 5% | | vehi- | | | | 10 |
| activity | basal | CO₂ | basal | cle | basal | 1 mg/kg | 3 mg/kg | mg/kg |
| Mean | 140 | 209 | 146 | 178 | 151 | 253 | 279 | 291 |
| SEM | 8 | 13 | 7 | 11 | 7 | 16 | 20 | 26 |
| p < | | — | | | | 0.001 | 0.001 | 0.001 |
| vs. | | | | | | | | |
| vehicle | | | | | | | | |

The compounds of example 1 (R-enantiomer) and of example 2 stimulate both the electromyographic activity of the genioglossus muscle (table 2 and 4), which increases the muscle tone of the upper respiratory pathways, and the respiratory minute volume (table 3 and 5) significantly. The increase in the muscle tone and the respiration-stimulation action prevents respiratory disorders, for example central or obstructive sleep apneas and snoring.

### F) Investigation of Compatibility in Rats

In the case of oral administration of 1000 mg/kg of the compound of example 1 (R-enantiomer), no side-effects were observed.

What is claimed is:

1. A method for the therapy or prophylaxis of a disease state selected from the group consisting of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, acute and chronic lung disorders with hypoxia and hypercapnia, neurodegenerative disorders, dementia, Alzheimer's disease, Parkinson's disease, Huntington's disease, cancer disorders, breast cancer, lung cancer, colon cancer and prostate cancer, the method comprising the administration of a therapeutically effective dose of a Kv1.5 inhibitor.

2. The method as claimed in claim 1 wherein the Kv1.5 inhibitor is selected from the group consisting of compounds of the formula Ia 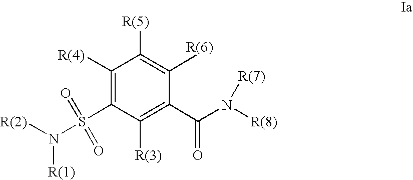
where R(8) is either a 1-indanyl radical of the formula II or a 2-indanyl radical of the formula III 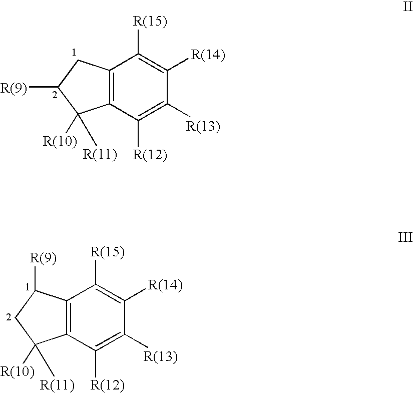
and in which:
R(1) and R(2)
are each independently R(20)-CᵣH₂ᵣ
where one CH₂ group of the CᵣH₂ᵣ group may be replaced by —O—, —CH═CH—, —C═C—, —CO—, —CO—O—, —O—CO—, —S—, —SO—, —SO₂—, —NR(21)—or —CONR(21);
R(21) is hydrogen or alkyl having 1, 2 or 3 carbon atoms;
R(20) is H, CH₃, CH₂F, CHF₂, CF₃, C₂F₅, C₃F₇, cycloalkyl having 3, 4, 5, 6, 7 or 8 carbon atoms, NR(22)R(23), —CONR(22)R(23), —OR(24), —COOR(24), phenyl or an N-containing heterocycle having 1, 2, 3, 4, 5, 6, 7, 8 or 9 carbon atoms,
where phenyl and the N-containing heterocycle are unsubstituted or substituted by one or two substituents selected from the group consisting of F, Cl, Br, I, CF₃, NO₂, CN, NH₂, OH, methyl, ethyl, hydroxymethyl, hydroxyethyl, methoxy, dimethylamino, sulfamoyl, methylsulfonyl and methylsulfonylamino;
R(22) and R(23)
are each independently hydrogen or alkyl having 1, 2, 3 or 4 carbon atoms;
or
R(22) and R(23)
together are a chain of 4 or 5 methylene groups of which one CH₂ group may be replaced by —O—, —S—, —NH—, —N(methyl)- or —N(benzyl)-;
R(24) is hydrogen or alkyl having 1, 2 or 3 carbon atoms;
r is zero, 1, 2, 3, 4, 5, 6, 7 or 8;
or
R(1) and R(2)
together are a chain of 4 or 5 methylene groups of which one CH₂ group may be replaced by —O—, —S—, —NH—, —N(methyl)- or —N(benzyl)-;
R(3), R(4), R(5) and R(6)
are each independently hydrogen, F, Cl, Br, I, alkyl having 1, 2, 3, 4 or 5 carbon atoms, cycloalkyl having 3, 4, 5, 6, 7 or 8 carbon atoms, CN, CF₃, NO₂, OR(25) or NR(26)R(27);
R(25) is hydrogen, alkyl having 1, 2, 3 or 4 carbon atoms, a fluorinated alkyl radical of the formula —CₓH₂ₓCF_{y}H_{3y} or phenyl,
which is unsubstituted or substituted by one or two substituents selected from the group consisting of F, Cl, Br, I, CF₃, NO₂, CN, NH₂, OH, methyl, ethyl, methoxy, dimethylamino, sulfamoyl, methylsulfonyl and methylsulfonylamino;
x is 0, 1, 2 or 3;
y is 1, 2 or 3;
R(26) and R(27)
are each independently hydrogen or alkyl having 1, 2, 3 or 4 carbon atoms;
or
R(26) and R(27)
together are a chain of 4 or 5 methylene groups of which one CH₂ group may be replaced by —O—, —S—, —NH—, —N(methyl)- or —N(benzyl)-;
R(7) is hydrogen or alkyl having 1, 2, 3 or 4 carbon atoms;
R(9) is hydrogen, OR(28) or OCOR(28);
R(28) is hydrogen or alkyl having 1, 2, 3 or 4 carbon atoms;
R(10) and R(11)
are each independently hydrogen or alkyl having 1, 2, 3 or 4 carbon atoms;
R(12), R(13), R(14) and R(15)
are each independently hydrogen, F, Cl, Br, I, alkyl having 1, 2, 3, 4 or 5 carbon atoms, cycloalkyl having 3, 4, 5, 6, 7 or 8 carbon atoms, —CN, —CF₃, —C₂F₅, —C₃F₇, —N₃, —NO₂, —Y—CₛH₂ₛ—R(29), phenyl, thienyl, furyl or an N-containing heterocycle having 1, 2, 3, 4, 5, 6, 7, 8 or 9 carbon atoms,
where phenyl, thienyl, furyl and the N-containing heterocycle are unsubstituted or substituted by one or two substituents selected from the group consisting of F, Cl, Br, I, CF₃, NO₂, CN, NH₂, OH, methyl, ethyl, methoxy, dimethylamino, sulfamoyl, methylsulfonyl and methylsulfonylamino;
Y is —O—, —CO—, —CO—O—, —O—CO—, —S—, —SO—, —SO₂—, —O—SO₂—, —SO₂NR(30)-, —CONR(30)- or —NR(30)CO—, where the bond to the base structure is in each case via the atom on the left;
R(30) is hydrogen or alkyl having 1, 2 or 3 carbon atoms;
s is zero, 1, 2, 3, 4, 5 or 6;
R(29) is hydrogen, methyl, CF₃, C₂H₅, C₃F₇, cycloalkyl having 3, 4, 5, 6, 7 or 8 carbon atoms, —OR(31), —COOR(31), —NR(32)R(33), —CONR(32)R(33), phenyl or an N-containing heterocycle having 1, 2, 3, 4, 5, 6, 7, 8 or 9 carbon atoms;
where phenyl and the N-containing heterocycle are unsubstituted or substituted by one or two substituents selected from the group consisting of F, Cl, Br, I, CF₃, NO₂, CN, NH₂, OH, methyl, ethyl, methoxy, dimethylamino, sulfamoyl, methylsulfonyl and methylsulfonylamino;
R(31) is hydrogen or alkyl having 1, 2 or 3 carbon atoms;
R(32) and R(33)
are each independently hydrogen or alkyl having 1, 2, 3 or 4 carbon atoms;
or
R(32) and R(33)
together are a chain of 4 or 5 methylene groups of which one CH₂ group may be replaced by —O—, —S—, —NH—, —N(CH₃)— or —N(benzyl)-;
compounds of the formula Ib 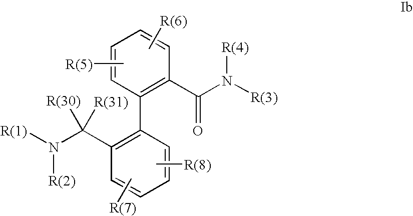
in which:
R(1) is C(O)OR(9), SO₂R(10), COR(11), C(O)NR(12)R(13) or C(S)NR(12)R(13);
R(9) is CₓH₂ₓ—R(14);
x is 0, 1, 2, 3 or 4,
where x cannot be zero when R(14) is OR(15) or SO₂Me;
R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
where phenyl, naphthyl, diphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is alkyl having 1, 2, 3, 4 or 5 carbon atoms, cycloalkyl having 3, 4, 5 or 6 carbon atoms, CF₃ or phenyl
which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(10), R(11) and R(12)
are each independently as defined for R(9);
R(13) is hydrogen, alkyl having 1, 2, 3 or 4 carbon atoms or CF₃;
R(2) is hydrogen, alkyl having 1, 2, 3 or 4 carbon atoms or CF₃;
R(3) is C_{y}H_{2y}—R(16);
y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or
R(3) is CHR(18)R(19);
R(18) is hydrogen or C_{z}H_{2z}—R(16) where R(16) is as defined above;
z is 0, 1, 2 or 3;
R(19) is COOH, CONH₂, CONR(20)R(21), COOR(22), CH₂OH;
R(20) is hydrogen, alkyl having 1, 2, 3, 4 or 5 carbon atoms, CᵥH₂ᵥ—CF₃ or C_{w}H_{2w}-phenyl,
where the phenyl ring is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
v is 0, 1, 2 or 3;
w is 0, 1, 2 or 3;
R(21) is hydrogen or alkyl having 1, 2, 3, 4 or 5 carbon atoms;
R(22) is alkyl having 1, 2, 3, 4 or 5 carbon atoms;
R(4) is hydrogen, alkyl having 1, 2, 3, 4, 5 or 6 carbon atoms or CF₃;
or
R(3) and R(4)
together are a chain of 4 or 5 methylene groups of which one methylene group may be replaced by —O—, —S—, —NH—, —N(methyl)- or —N(benzyl)-;
R(5), R(6), R(7) and R(8)
are each independently hydrogen, F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;
R(30) and R(31)
are each independently hydrogen or alkyl having 1, 2 or 3 carbon atoms;
or
R(30) and R(31)
together are a chain of 2 methylene groups;
compounds of the formula Ic 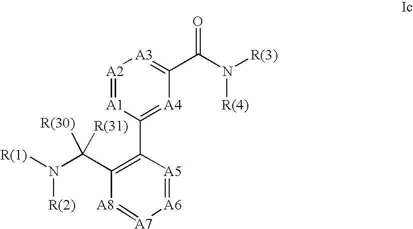
in which:
A1, A2, A3, A4, A5, A6, A7 and A8
are each independently nitrogen, CH or CR5, where at least four of these groups are CH;
R(1) is C(O)OR(9), SO₂R(10), COR(11), C(O)NR(12)R(13) or C(S)NR(12)R(13);
R(9), R(10), R(11) and R(12)
are each independently CₓH₂ₓ—R(14);
x is 0, 1, 2, 3 or 4;
where x cannot be 0 when R(14) is OR(15) or SO₂Me;
R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
where phenyl, naphthyl, biphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is alkyl having 1, 2, 3, 4 or 5 carbon atoms, cycloalkyl having 3, 4, 5 or 6 carbon atoms, CF₃ or phenyl
which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
R(2) is hydrogen, alkyl having 1, 2, 3 or 4 carbon atoms or CF₃;
R(3) is C_{y}H_{2y}—R(16);
y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino,
R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or
R(3) is CHR(18)R(19);
R(18) is hydrogen or C_{z}H_{2z}—R(16) where R(16) is as defined above;
z is 0, 1, 2 or 3;
R(19) is COOH, CONH₂, CONR(20)R(21), COOR(22) or CH₂OH;
R(20) is hydrogen, alkyl having 1, 2, 3, 4 or 5 carbon atoms, CᵥH₂ᵥ—CF₃ or C_{w}H_{2w}-phenyl
where the phenyl ring is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
v is 0, 1, 2 or 3;
w is 0, 1, 2 or 3;
R(21) is hydrogen or alkyl having 1, 2, 3, 4 or 5 carbon atoms;
R(22) is alkyl having 1, 2, 3, 4 or 5 carbon atoms;
R(4) is hydrogen, alkyl having 1, 2, 3, 4, 5 or 6 carbon atoms or CF₃;
or
R(3) and R(4)
together are a chain of 4 or 5 methylene groups of which one methylene group may be replaced by —O—, —S—, —NH—, —N(methyl)- or —N(benzyl)-;
R(5) is F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino, where, in the case that a plurality of A1 to A8 radicals is defined as CR(5), the R(5) radicals are each defined independently;
R(30) and R(31)
are each independently hydrogen or alkyl having 1, 2 or 3 carbon atoms;
or
R(30) and R(31)
together are a chain of 2 methylene groups;
compounds of the formula Id 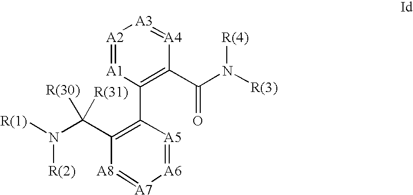
in which:
A1, A2, A3, A4, A5, A6, A7 and A8 are each independently nitrogen, CH or CR(5), where at least one of these groups is nitrogen and at least 4 of these groups are CH;
R(1) is C(O)OR(9), SO₂R(10), COR(11), C(O)NR(12)R(13) or C(S)NR(12)R(13);
R(9), R(10), R(11) and R(12)
are each independently CₓH₂ₓ—R(14);
x is 0, 1, 2, 3 or 4;
where x cannot be 0 when R(14) is OR(15) or SO₂Me;
R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
where phenyl, naphthyl, biphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is alkyl having 1, 2, 3, 4 or 5 carbon atoms, cycloalkyl having 3, 4, 5 or 6 carbon atoms, CF₃ or phenyl which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
R(2) is hydrogen, alkyl having 1, 2, 3 or 4 carbon atoms or CF₃;
R(3) is C_{y}H_{2y}—R(16);
y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino,
R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or
R(3) is CHR(18)R(19);
R(18) is hydrogen or C_{z}H_{2z}—R(16) where R(16) is as defined above;
z is 0, 1, 2 or 3;
R(19) is COOH, CONH₂, CONR(20)R(21), COOR(22) or CH₂OH;
R(20) is hydrogen, alkyl having 1, 2, 3, 4 or 5 carbon atoms, CᵥH₂ᵥ—CF₃ or C_{w}H_{2w}-phenyl
where the phenyl ring is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
v is 0, 1, 2 or 3;
w is 0, 1, 2 or 3;
R(21) is hydrogen or alkyl having 1, 2, 3, 4 or 5 carbon atoms;
R(22) is alkyl having 1, 2, 3, 4 or 5 carbon atoms;
R(4) is hydrogen, alkyl having 1, 2, 3, 4, 5 or 6 carbon atoms or CF₃;
or
R(3) and R(4)
together are a chain of 4 or 5 methylene groups of which one methylene group may be replaced by —O—, —S—, —NH—, —N(methyl)- or —N(benzyl)-;
R(5) are each independently F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino, where, in the case that a plurality of A1 to A8 radicals is defined as CR(5), the R(5) radicals are each defined independently;
R(30) and R(31)
are each independently hydrogen or alkyl having 1, 2 or 3 carbon atoms;
or
R(30) and R(31)
together are oxygen or a chain of 2 methylene groups;
compounds of the formula Ie or If 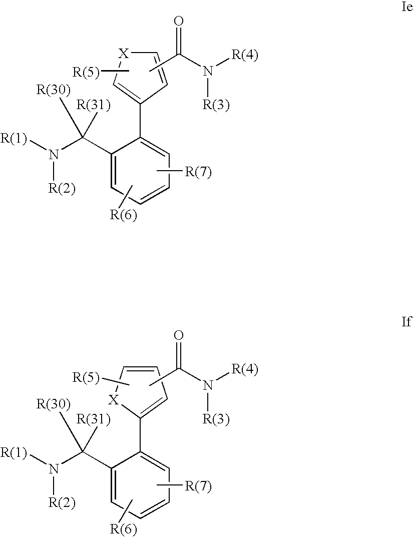
in which:
X is oxygen or sulfur;
R(1) is C(O)OR(9), SO₂R(10), COR(11), C(O)NR(12)R(13) or C(S)NR(12)R(13);
R(9), R(10), R(11) and R(12) are each independently CₓH₂ₓ—R(14);
x is 0, 1, 2, 3 or 4;
where x cannot be 0 when R(14) is OR(15) or SO₂Me;
R(14) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(15), SO₂Me, phenyl, naphthyl, biphenylyl, furyl, thienyl or an N-containing heteroaromatic having 1, 2, 3, 4, 5, 6, 7, 8 or 9 carbon atoms,
where phenyl, naphthyl, biphenylyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is alkyl having 1, 2, 3, 4 or 5 carbon atoms, cycloalkyl having 3, 4, 5 or 6 carbon atoms, CF₃ or phenyl which is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is hydrogen, alkyl having 1, 2, 3 or 4 carbon atoms or CF₃;
R(2) is hydrogen, alkyl having 1, 2, 3 or 4 carbon atoms or CF₃;
R(3) is C_{y}H_{2y}—R(16);
y is 0, 1, 2, 3 or 4,
where y cannot be 0 when R(16) is OR(17) or SO₂Me;
R(16) is alkyl having 1, 2, 3, 4, 5 or 6 carbon atoms, cycloalkyl having 3, 4, 5, 6, 7, 8, 9, 10 or 11 carbon atoms, CF₃, C₂F₅, C₃F₇, CH₂F, CHF₂, OR(17), SO₂Me, phenyl, naphthyl, furyl, thienyl or an N-containing heteroaromatic having 1, 2, 3, 4, 5, 6, 7, 8 or 9 carbon atoms,
where phenyl, naphthyl, furyl, thienyl and the N-containing heteroaromatic are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino,
R(17) is hydrogen, alkyl having 1, 2, 3, 4 or 5 carbon atoms, cycloalkyl having 3, 4, 5 or 6 carbon atoms, CF₃, phenyl or 2-, 3- or 4-pyridyl,
where phenyl or 2-, 3- or 4-pyridyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
or
R(3) is CHR(18)R(19);
R(18) is hydrogen or C_{z}H_{2z}—R(16) where R(16) is as defined above;
z is 0, 1, 2 or 3;
R(19) is COOH, CONH₂, CONR(20)R(21), COOR(22) or CH₂OH;
R(20) is hydrogen, alkyl having 1, 2, 3, 4 or 5 carbon atoms, CᵥH₂ᵥ—CF₃ or C_{w}H_{2w}-phenyl
where the phenyl ring is unsubstituted or substituted by 1, 2 or 3 substituents selected from the group consisting of F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
v is 0, 1, 2 or 3;
w is 0, 1, 2 or 3;
R(21) is hydrogen or alkyl having 1, 2, 3, 4 or 5 carbon atoms;
R(22) is alkyl having 1, 2, 3, 4 or 5 carbon atoms;
R(4) is hydrogen, alkyl having 1, 2, 3, 4, 5 or 6 carbon atoms or CF₃;
or
R(3) and R(4)
together are a chain of 4 or 5 methylene groups of which one methylene group may be replaced by —O—, —S—, —NH—, —N(methyl)- or —N(benzyl)-;
R(5), R(6) and R(7)
are each independently hydrogen, F, Cl, Br, I, CF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl or methylsulfonylamino;
R(30) and R(31)
are each independently hydrogen or alkyl having 1, 2 or 3 carbon atoms;
or
R(30) and R(31)
together are oxygen or a chain of 2 methylene groups;
compounds of the formula Ig 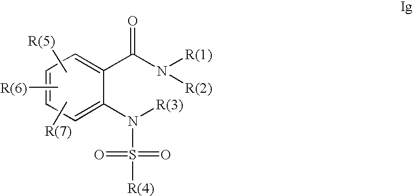
in which
R(1) is (CH₂)ₓ—R(8)
x is 0, 1, 2, 3, 4 or 5,
R(8) is phenyl, thienyl or furanyl,
where phenyl, thienyl and furanyl are unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(2) is hydrogen or alkyl having 1, 2 or 3 carbon atoms;
R(3) is hydrogen or alkyl having 1, 2 or 3 carbon atoms;
R(4) is alkyl having 3, 4, 5, 6 or 7 carbon atoms, cycloalkyl having 3, 4, 5, 6 or 7 carbon atoms, phenyl, naphthyl or heteroaryl,
where phenyl and heteroaryl are unsubstituted or substituted by 1, 2 or 3 substituents selected from the group consisting of F, Cl, Br, I, CF₃, OCF₃, NO₂, COOMe, CONH₂, COMe, NH₂, OH, alkyl having 1, 2, 3 or 4 carbon atoms, alkoxy having 1, 2, 3 or 4 carbon atoms, dimethylamino, methylsulfonyl and methylsulfonylamino;
R(5), R(6) and R(7)
are each independently F, Cl, Br, I, CF₃, OCF₃, NO₂, CN, COOMe, CONH₂, COMe, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
compounds of the formula Ih 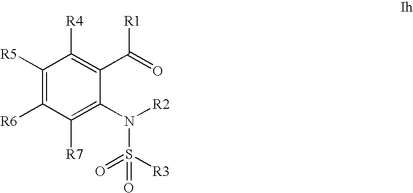
in which: 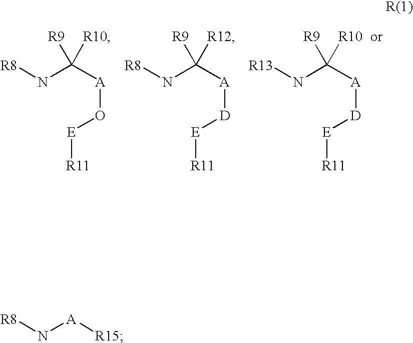
A is —CₙH₂ₙ—;
n is 0, 1, 2, 3, 4 or 5;
O is oxygen;
D is a bond or oxygen;
E is —CₘH₂ₘ—;
m is 0, 1, 2, 3, 4 or 5;
R(8) is hydrogen, alkyl having 1, 2, 3 or 4 carbon atoms or CₚH₂ₚ—R(14);
p is 0, 1, 2, 3, 4 or 5;
R(14) is cycloalkyl having 3, 4, 5 or 6 carbon atoms, aryl or heteroaryl,
where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(9) is hydrogen or alkyl having 1, 2, 3, 4, 5 or 6 carbon atoms;
R(10) is hydrogen, alkyl having 1, 2, 3 or 4 carbon atoms, cycloalkyl having 3, 4, 5 or 6 carbon atoms, aryl or heteroaryl,
where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(11) is cycloalkyl having 3, 4, 5 or 6 carbon atoms, phenyl, naphthyl, thienyl, furyl, pyridyl, pyrazinyl, pyridazinyl or pyrimidyl,
where phenyl, naphthyl, thienyl, furyl, pyridyl, pyrazinyl, pyridazinyl and pyrimidyl are each unsubstituted or substituted by 1, 2 or 3 substituents selected from the group consisting of F, Cl, Br, I, CF₃, OCF₃, NO₂, CN, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(12) is alkyl having 1, 2, 3 or 4 carbon atoms, cycloalkyl having 3, 4, 5 or 6 carbon atoms, aryl or heteroaryl,
where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is CₚH₂ₚ—R(14′);
p is 0, 1, 2, 3, 4 or 5;
R(14′) is cycloalkyl having 3, 4, 5 or 6 carbon atoms, tetrahydrofuranyl, tetrahydropyranyl, aryl or heteroaryl,
where aryl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(15) is cycloalkyl having 3, 4, 5, 6, 7 or 8 carbon atoms;
R(2) is hydrogen or alkyl having 1, 2, 3 or 4 carbon atoms;
R(3) is alkyl having 3, 4, 5, 6 or 7 carbon atoms, cycloalkyl having 3, 4, 5 or 6 carbon atoms, phenyl or naphthyl,
where phenyl or naphthyl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(4), R(5), R(6) and R(7)
are each independently hydrogen, F, Cl, Br, I, CF₃, OCF₃, OCHF₂, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
compounds of the formula Ij 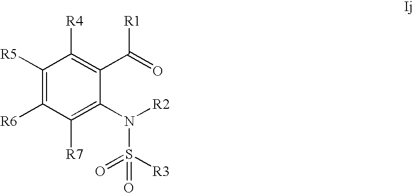
in which: 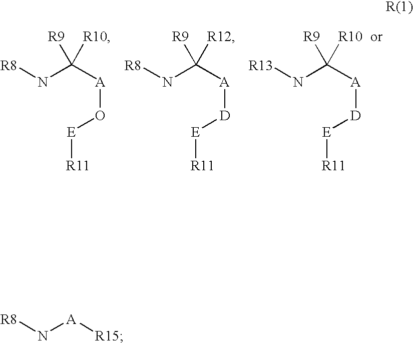
A is —CₙH₂ₙ—;
n=0, 1, 2, 3, 4 or 5;
D is a bond or —O—;
E is —Cₘ2₂ₘ—;
m=0, 1, 2, 3, 4 or 5;
R(8) is hydrogen, alkyl having 1, 2, 3 or 4 carbon atoms or CₚH₂ₚ—R(14);
p is 0, 1, 2, 3, 4 or 5;
R(14) is phenyl, naphthyl or heteroaryl,
where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(9) is hydrogen or alkyl having 1, 2, 3, 4, 5 or 6 carbon atoms;
R(10) is hydrogen, alkyl having 1, 2, 3 or 4 carbon atoms, phenyl, naphthyl or heteroaryl,
where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(11) is cycloalkyl having 3, 4, 5 or 6 carbon atoms, phenyl, naphthyl, thienyl, furyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl,
where phenyl, naphthyl, thienyl, furyl, pyridyl, pyrazinyl, pyrimidinyl, pyridazinyl, indolyl, indazolyl, quinolyl, isoquinolyl, phthalazinyl, quinoxalinyl, quinazolinyl or cinnolinyl are each unsubstituted or substituted by 1, 2 or 3 substituents selected from the group consisting of F, Cl, Br, I, CF₃, OCF₃, NO₂, CN, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(12) is alkyl having 1, 2, 3 or 4 carbon atoms, alkynyl having 1, 2, 3 or 4 carbon atoms, cycloalkyl having 3, 4, 5 or 6 carbon atoms, phenyl, naphthyl or heteroaryl,
where phenyl, naphthyl and heteroaryl are each unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(13) is CₚH₂ₚ—R(14);
p is 0, 1, 2, 3, 4 or 5;
R(15) is cycloalkyl having 3, 4, 5, 6, 7 or 8 carbon atoms;
R(2) is hydrogen or alkyl having 1, 2, 3 or 4 carbon atoms;
R(3) is heteroaryl,
where heteroaryl is unsubstituted or substituted by 1, 2 or 3 substituents selected from the group consisting of F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
R(4), R(5), R(6) and R(7)
are each independently hydrogen, F, Cl, Br, I, CF₃, OCF₃, NO₂, CN, COOMe, CONH₂, COMe, NH₂, OH, alkyl having 1, 2, 3 or 4 carbon atoms, alkoxy having 1, 2, 3 or 4 carbon atoms, dimethylamino, sulfamoyl, methylsulfonyl and methylsulfonylamino;
and combinations thereof,
or physiologically compatible salts of the compounds of the formula Ia, Ib, Ic, Id, Ie, If, Ig, Ih or Ij, or combinations thereof.

3. The method as claimed in claim 1 wherein the disease state is selected from the group consisting of respiratory disorders, sleep-related respiratory disorders, central and obstructive sleep apneas, upper airway resistance syndrome, Cheyne-Stokes respiration, snoring, disrupted central respiratory drive, sudden child death, postoperative hypoxia and apnea, muscle-related respiratory disorders, respiratory disorders after long-term ventilation, respiratory disorders during adaptation in high mountains, and acute and chronic lung disorders with hypoxia and hypercapnia.

4. The method as claimed in claim 1 wherein the disease state is selected from the group consisting of sleep-related respiratory disorders, central and obstructive sleep apneas, upper airway resistance syndrome and snoring.

5. The method as claimed in claim 1 wherein the disease state is selected from the group consisting of sleep-related respiratory disorders, central and obstructive sleep apneas and snoring.

6. The method as claimed in claim 1 wherein the disease state is sleep apneas.

7. The method as claimed in claim 1 wherein the disease state is selected from the group consisting of neurodegenerative disorders, dementia, Alzheimer's disease, Parkinson's disease and Huntington's disease.

8. The method as claimed in claim 1 wherein the disease state is selected from the group consisting of cancer disorders, breast cancer, lung cancer, colon cancer and prostate cancer.

9. The method of claim 2 wherein the compounds are selected from the group of
2-(Butyl-1-sulfonylamino)-N-[1(R)-(6-methoxypyridin-3-yl)propyl]benzamide,
2-(Butyl-1-sulfonylamino)-N-[1(S)-(6-methoxypyridin-3-yl)propyl]benzamide,
N-(2-Pyridin-3-ylethyl)-2′-{[2-(4-methoxyphenyl)acetylamino]methyl}biphenyl-2-carboxamide,
(S)-5-Fluoro-2-(quinoline-8-sulfonylamino-N-(1-phenylpropyl)benzamide,
(S)-5-Methoxy-2-(4-methoxybenzenesulfonylamino)-N-(1-phenylpropyl)benzamide,
N-(2-(R)-hydroxypropyl)-2′-(α-(S)-methylbenzyloxycarbonylaminomethyl)biphenyl-2-carboxamide,
N-(2,4-Difluorobenzyl)-5-chloro-2′-{[2-(4-methoxyphenyl)acetylamino]methyl}biphenyl-2-carboxamide,
Benzyl {2′-[methyl(2-pyridin-2-ylethyl)carbamoyl]biphenyl-2-ylmethyl}carbamate
N-(2,4-Difluorobenzyl)-3-(2-{[2-(4-methoxyphenyl)acetylamino]methyl}-phenyl)thiophene-2-carboxamide,
N-(2,4-Difluorobenzyl)-5-(2-{[2-(4-methoxyphenyl)acetylamino]methyl}phenyl)furan-2-carboxamide,
N-(3-Methylbutyl)-2-(2-{[2-(4-methoxyphenyl)acetylamino]methyl}phenyl)furan-3-carboxamide,
N-(2,4-Difluorobenzyl)-2-(2-{[2-(4-methoxyphenyl)acetylamino]methyl}-phenyl)thiophene-3-carboxamide,
(S)-1-Phenylethyl {2-[2-(2-pyridin-2-ylethylcarbamoyl)pyridin-3-yl]benzyl}carbamate,
N-Cyclopropylmethyl-2-{2-[((R)-3-phenylbutyrylamino)methyl]phenyl}pyridine-2-carboxamide,
Benzyl {2-[3-(2,4-difluorobenzylcarbamoyl)pyridin-2-yl]benzyl}carbamate,
Benzyl {4-[3-(3-methylbutylcarbamoyl)phenyl]pyridin-3-ylmethyl}carbamate,
N-(3-Methylbutyl)-2′-{[3-(4-methoxyphenyl)propionylamino]methyl}-6-methylbiphenyl-3-carboxamide,
and N-indan-1-yl-2-methyl-5-(3-methylbutylsulfamoyl)benzamide,
or physiologically compatible salts thereof.

10. The method of claim 1 wherein the Kv1.5 inhibitor is administered by intravenous administration.

11. The method of claim 1 wherein the Kv1.5 inhibitor is administered by oral administration.

12. The method of claim 1 wherein the Kv1.5 inhibitor is administered by nasal administration.
